(19) United States
(12) Patent Application Publication
(10) Pub. No.: US 2024/0087750 A1
(43) Pub. Date: Mar. 14, 2024

(54) MACHINE LEARNING SYSTEMS AND METHODS FOR PREDICTING RISK OF INCIDENT OPIOID USE DISORDER AND OPIOID OVERDOSE

(71) Applicants: University of Florida Research Foundation, Incorporated, Gainesville, FL (US); University of Pittsburgh- Of the Commonwealth System of Higher Education, Pittsburgh, PA (US); The United States Government as represented by The Department of Veterans Affairs, Washington, DC (US)

(72) Inventors: Wei Hsuan Lo Ciganic, Gainesville, FL (US); Walid Fouad Gellad, Pittsburgh, PA (US)

(73) Assignees: University of Florida Research Foundation, Incorporated, Gainesville, FL (US); University of Pittsburgh- Of the Commonwealth System of Higher Education, Pittsburgh, PA (US); The United States Government as represented by The Department of Veterans Affairs, Washington, DC (US)

(21) Appl. No.: 18/010,083

(22) PCT Filed: Jun. 17, 2021
(86) PCT No.: PCT/US21/37743

§ 371 Date: Dec. 13, 2022

Related U.S. Application Data
(60) Provisional application No. 63/040,451, filed on Jun. 17, 2020.

(51) Int. Cl. G16H 50/30; G16H 20/10; G16H 50/20

(52) U.S. Cl. CPC G16H 50/30; G16H 20/10; G16H 50/20

(57) ABSTRACT

h A method for using a trained machine learning model to predict risk of incident opioid use disorder (OUD) and/or of N an opioid overdose episode for a subject. The method comprises using at least one computer hardware processor to perform: accessing data associated with the subject, wherein the data comprises values for a plurality of predictors; generating input features for the trained machine learning model from the data; and providing the input features as input to the trained machine learning model to obtain an output indicative of the risk of OUD and/or of the opioid overdose episode for the subject, wherein the trained machine learning model comprises a first plurality of values for a respective first plurality of parameters, the first plurality of values used by the at least one computer hardware processor to obtain the output from the input features.

### RELATED APPLICATIONS

This application is a national stage filing under 35 U.S.C. § 371 of International Patent Application Serial No. PCT/US2021/037743, filed Jun. 17, 2021, entitled “MACHINE LEARNING SYSTEMS AND METHODS FOR PREDICTING RISK OF INCIDENT OPIOID USE DISORDER AND OPIOID OVERDOSE”, which claims the benefit under 35 U.S.C. § 119(e) of Provisional Patent App. No. 63/040,451 entitled “MACHINE LEARNING SYSTEMS AND METHODS FOR PREDICTING RISK OF INCIDENT OPIOID USER DISORDER AND/OR AN OPIOID OVERDOSE EPISODE,” filed on Jun. 17, 2020, which are incorporated by reference herein in their entirety.

### FEDERALLY SPONSORED RESEARCH

This invention was made with government support under grant numbers RO1 DA044985 and R21 AG060308 awarded by the National Institutes of Health and I01 HX002389-01 awarded by Health Services Research and Development Service (HSR&D)— Department of Veterans Affairs (VA). The government has certain rights in the invention.

### FIELD

The present disclosure relates generally to machine learning techniques for predicting whether a patient is at risk for being diagnosed with incident opioid use disorder (OUD) and/or is at risk of having an opioid overdose episode.

### BACKGROUND

Millions of individuals in America have reported using prescription opioids nonmedically. Furthermore, an estimated 115 individuals die each day from opioid overdose. The annual cost of misuse or abuse of opioids exceeds $78.5 billion, including cost of health care, lost productivity, substance abuse treatment, and costs to the criminal justice system.

### SUMMARY

Some embodiments provide for a method for using a trained machine learning model to predict risk of incident opioid use disorder (OUD) and/or of an opioid overdose episode for a subject, the method comprising: using at least one computer hardware processor to perform: accessing data associated with the subject, wherein the data comprises values for at least 10 predictors from among predictors shown in Table 1 and/or Table 2; generating input features for the trained machine learning model from the data; and providing the input features as input to the trained machine learning model to obtain an output indicative of the risk of OUD and/or of the opioid overdose episode for the subject, wherein the trained machine learning model comprises a first plurality of values for a respective first plurality of parameters, the first plurality of values used by the at least one computer hardware processor to obtain the output from the input features.

Some embodiments provide a system for using a trained machine learning model to predict risk of incident opioid use disorder (OUD) and/or of an opioid overdose episode for a subject, the system comprising: at least one computer hardware processor; and at least one non-transitory computer-readable storage medium storing instructions that, when executed by the at least one computer hardware processor, cause the at least one computer hardware processor to perform: accessing data associated with the subject, wherein the data comprises values for at least 10 predictors from among predictors shown in Table 1 and/or Table 2; generating input features for the trained machine learning model from the data; and providing the input features as input to the trained machine learning model to obtain an output indicative of the risk of OUD and/or the opioid overdose episode for the subject, wherein the trained machine learning model comprises a first plurality of values for a respective first plurality of parameters, the first plurality of values used by the at least one computer hardware processor to obtain the output from the input features.

Some embodiments provide for at least one non-transitory computer-readable storage medium storing instructions that, when executed by at least one computer hardware processor, cause the at least one computer hardware processor to perform: accessing data associated with a subject, wherein the data comprises values for at least 10 predictors from among predictors shown in Table 1 and/or Table 2; generating input features for a trained machine learning model from the data; and providing the input features as input to the trained machine learning model to obtain an output indicative of a risk of OUD and/or of an opioid overdose episode for the subject, wherein the trained machine learning model comprises a first plurality of values for a respective first plurality of parameters, the first plurality of values used by the at least one computer hardware processor to obtain the output from the input features.

Some embodiments provide for a method for using a trained machine learning model to predict risk of incident opioid use disorder (OUD) and/or of an opioid overdose episode for a subject, the method comprising: using at least one computer hardware processor to perform: accessing data associated with the subject, wherein the data comprises values for one or more social determinants of health; generating input features for the trained machine learning model from the data; and providing the input features as input to the trained machine learning model to obtain an output indicative of the risk of OUD and/or of the opioid overdose episode for the subject, wherein the trained machine learning model comprises a first plurality of values for a respective first plurality of parameters, the first plurality of values used by the at least one computer hardware processor to obtain the output from the input features.

Some embodiments provide a system for using a trained machine learning model to predict risk of incident opioid use disorder (OUD) and/or of an opioid overdose episode for a subject, the system comprising: at least one computer hardware processor; and at least one non-transitory computer-readable storage medium storing instructions that, when executed by the at least one computer hardware processor, cause the at least one computer hardware processor to perform: accessing data associated with the subject, wherein the data comprises values for one or more social determinants of health; generating input features for the trained machine learning model from the data; and providing the input features as input to the trained machine learning model to obtain an output indicative of the risk of OUD and/or of the opioid overdose episode for the subject, wherein the trained machine learning model comprises a first plurality of values for a respective first plurality of parameters, the first plurality of values used by the at least one computer hardware processor to obtain the output from the input features.

Some embodiments provide for at least one non-transitory computer-readable storage medium storing instructions that, when executed by at least one computer hardware processor, cause the at least one computer hardware processor to perform: accessing data associated with a subject, wherein the data comprises values for one or more social determinants of health; generating input features for a trained machine learning model from the data; and providing the input features as input to the trained machine learning model to obtain an output indicative of a risk of OUD and/or of an opioid overdose episode for the subject, wherein the trained machine learning model comprises a first plurality of values for a respective first plurality of parameters, the first plurality of values used by the at least one computer hardware processor to obtain the output from the input features.

### BRIEF DESCRIPTION OF THE DRAWINGS

Various aspects and embodiments will be described herein with reference to the following figures. It should be appreciated that the figures are not necessarily drawn to scale. Items appearing in multiple figures are indicated by the same or a similar reference number in all the figures in which they appear.

FIG. 1A is a diagram illustrating an example opioid use management system, according to some embodiments of the technology described herein.

FIG. 1B is a diagram illustrating example operation of the prediction system of the opioid use management system of FIG. 1A, according to some embodiments of the technology described herein.

FIG. 1C is a diagram illustrating an example training system for training a machine learning model, according to some embodiments of the technology described herein.

FIG. 2 is a diagram illustrating an example process of using a trained machine learning model to predict risk of OUD and/or of an opioid overdose episode, according to some embodiments of the technology described herein.

FIG. 3 is a diagram illustrating an example timeline of operation of an opioid use management system, according to some embodiments of the technology described herein.

FIG. 4 is a diagram illustrating an example process of training a machine learning model to predict risk of OUD and/or of an opioid overdose episode, according to some embodiments of the technology described herein.

FIG. 5 is an example graphical user interface (GUI) showing a prediction of risk of an opioid overdose episode for a set of subjects, according to some embodiments of the technology described herein.

FIG. 6 is an example GUI showing a prediction of risk of an opioid overdose episode for a subject and recommended treatments, according to some embodiments of the technology described herein.

FIG. 7 is an example GUI showing a prediction of risk of opioid overdose and OUD for a subject, according to some embodiments of the technology described herein.

FIG. 8 is a plot of a distribution of subjects based on predicted risk of OUD, according to some embodiments of the technology described herein.

FIG. 9 is a plot of a set of 25 predictors identified for prediction of risk of OUD in a machine learning model trained using Elastic Net (EN) regularization, according to some embodiments of the technology described herein.

FIG. 10 is a plot of an example set of features determined to contribute to a prediction of risk of OUD in a subject, according to some embodiments of the technology described herein.

FIG. 11 is a plot of an example set of features determined to contribute to a prediction of risk of OUD and/or and opioid overdose episode in a subject, according to some embodiments of the technology described herein.

FIG. 12 is a plot of opioid and benzodiazepine dosage patterns, according to some embodiments of the technology described herein.

FIG. 13 is a diagram of an example computer system, according to some embodiments of the technology described herein.

### DETAILED DESCRIPTION

The inventors have recognized that conventional approaches for determining whether a subject (e.g., a patient, a patient undergoing opioid therapy, or other individual) is at risk for being diagnosed with OUD and/or is at risk of having an opioid overdose episode are not accurate and may be improved. Conventional approaches use a small number of predictors in models that do not accurately predict a subject's risk of being diagnosed with OUD and/or of having an opioid overdose episode. For example, conventional approaches use individual risk factors such as dose of opioids prescribed to a subject to determine the subject's risk of overdose and whether to intervene in the subject's opioid therapy. As a result of inaccurately identifying high risk subjects, intervention resources (e.g., therapy, additional medication, enrollment in lock-in programs) are wasted on individuals who are not in need of any intervention, while not being used for those that require intervention.

To address shortcomings of conventional approaches, the inventors have developed machine learning techniques that more accurately and reliably predict risk of OUD and/or opioid overdose for subjects than conventional methods. In particular, the inventors have identified multiple predictors that, when used together with machine learning techniques, enable the machine learning techniques to provide more accurate predictions than previously possible with conventional prediction methods.

Accordingly, in some embodiments, the machine learning techniques use information about a subject accessed from one or more data sources (e.g., electronic health records (EHR), insurance claims data, etc.) to generate input features for a trained machine learning model (e.g., a logistic regression model, deep neural network model, a random forest model, and/or a gradient boosting machine model). The input features are provided as input to the trained machine learning model which, in turn, generates an output indicating the risk of OUD and/or the risk of an opioid overdose episode. The trained machine learning model may comprise multiple learned parameters that are used by a computing device to obtain the output using the input features. The output may indicate a risk of OUD and/or opioid overdose for a subject. For example, the output may indicate a likelihood (e.g., a probability) that the subject will develop OUD and/or an opioid overdose episode.

Given the improved accuracy of the techniques described herein, the techniques improve over conventional approaches by allowing care providers to identify and initiate preventative treatments to reduce potential harms resulting from development of OUD and/or an episode of opioid overdose. Some embodiments may use the output to determine whether to intervene in the subject's opioid therapy or address a risk of adverse events. If it is determined that the indicated risk warrants intervention, then intervention in the subject's opioid therapy may be initiated (e.g., by adjusting medication for the subject, enrolling the subject in a lock-in program, dispensing medication, and/or administering opioid antagonist therapy) and/or any other intervention known to decrease risk of overdose may be employed on the subject based on the risk (e.g., predicted likelihood or classification). Some embodiments may use the output to determine an evidence-based intervention for the subject. An evidence-based intervention may be a treatment that has been proven effective through outcomes of the treatment on other subjects. Example evidence-based interventions include a naloxone kit distribution, a structure of visits to healthcare providers, syringe service programs, or other evidence-based interventions.

Some embodiments of the technology described herein may be implemented in a software platform that provides care providers a tool for monitoring and treatment of subjects (e.g., patients prescribed opioid). In some embodiments, the machine learning techniques described herein may be implemented in software that may be used by care providers (e.g., clinicians) working with patients. For example, the techniques described herein may be implemented in conjunction with a software application, which may be a web-based application, a mobile application, or any other suitable software application. In some embodiments, the software application may include one or more graphical user interfaces (GUIs). For example, some embodiments generate a graphical user interface (GUI) that provide a care provider (e.g., a clinician) an indication of risk of OUD and/or an opioid overdose for a subject based on an output of a machine learning model indicating risk of OUD and/or the risk of an opioid overdose for the subject, possible causes of an indicated risk (e.g., factors that contributed to the prediction), and/or recommended adjustments in a subject's medication based on the output of the machine learning model indicating the risk of OUD and/or the risk of an opioid overdose for the subject.

The inventors have also recognized that social determinants of health improve the performance of machine learning techniques in predicting the risks of OUD and opioid overdose for subjects. Accordingly, in some embodiments, the machine learning techniques developed by the inventors integrate social determinants of health (e.g., data indicating economic stability, education, and/or community context). Such social determinants of health for a subject, may be used to generate input to a trained machine learning model to predict OUD and/or overdose risk for that subject. The inventors have demonstrated that incorporating these data improves the accuracy predictions of the risk of OUD and/or the risk of opioid overdose for a subject.

Some embodiments described herein address all the above-described issues that the inventors have recognized with conventional approaches for determining whether a subject is at risk for being diagnosed with OUD and/or is at risk of having an opioid overdose episode. However, it should be appreciated that not every embodiment described herein addresses every one of these issues. It should also be appreciated that embodiments of the technology described herein may be used for purposes other than addressing the above-described issues of conventional approaches for determining whether a subject is at risk for being diagnosed with OUD and/or is at risk of having an opioid overdose episode.

Accordingly, some embodiments provide for a method for using a trained machine learning model to predict risk of OUD and/or an opioid overdose episode for a subject (e.g., a subject without a history of OUD), the method comprising: (A) accessing data associated with the subject (e.g., from one or more databases), wherein the data comprises values for at least 10 predictors from among predictors shown in Table 1 and/or Table 2; (B) generating input features for the trained machine learning model from the data; and (C) providing the input features as input to the trained machine learning model to obtain an output indicative of the risk of OUD and/or of an opioid overdose episode for the subject, wherein the trained machine learning model comprises a first plurality of values for a respective first plurality of parameters, the first plurality of values used by the at least one computer hardware processor to obtain the output from the input features.

In some embodiments, the first plurality of parameters may comprise any suitable number of parameters including, for example, at least 10, at least 100, at least 1,000, at least 10,000, at least 100,000, at least 1,000,000, between 10 and 10,000, between 50 and 1,000,000 or any other suitable ranue within these ranges.

In some embodiments the trained machine learning model may be a non-linear regression model, for example, a logistic regression model. In some embodiments, non-linear regression model may be trained using a regularization technique (e.g., LASSO, Tikhonov, ridge regression, Elastic Net (EN) regularization). As another example, a neural network model (e.g., a deep neural network), a random forest model, and/or a gradient boosting model may be employed. The regularization may result in a model that utilizes a subset of candidate predictors. For example, in some embodiments a model having between 30 and 60 predictors (e.g., 48) may be obtained from training data that includes hundreds of candidate predictors.

In some embodiments, the machine learning model may be trained using training data and a supervised learning technique, wherein the training data comprises paired data comprising input-output pairs, each input-output pair having input values for the at least 10 predictors and a corresponding output value indicative of a risk of OUD and/or of an opioid overdose episode. In some embodiments, the corresponding output value indicative of the risk of OUD and/or of an opioid overdose episode is set based on an indication of OUD, opioid overdose diagnosis, and/or initiation of methadone or buprenorphine.

In some embodiments, the output from the trained machine learning model indicates the risk of OUD for the subject within a predetermined time period (e.g., 1 month, 2 months, 3 months, 4 months, 5 months, 6 months, etc.) of the subject receiving an opioid prescription. In some embodiments, updated data associated with the subject may be received (e.g., periodically) and the trained machine learning model may be used to obtain an updated estimate of risk. Receiving the updated data periodically may include receiving the updated data at regular time intervals. For example, the updated data associated with the subject may be received every day, week, month, 2 months, 3 months, 4 months, 5 months, 6 months, year, or at any other suitable frequency. In another example, the updated data associated with the subject may be received every k weeks, where k is an integer between 1 and 10. In another example, the updated data associated with the subject may be received every k months, where k is an integer between 1 and 12.

In some embodiments, the data associated with the subject comprises values for at least some (e.g., at least one, at least three, at least five, at least ten) predictors from “patterns of prescription opioid use” predictors listed in Table 1. In some embodiments, the data associated with the subject comprises values for at least some (e.g., at least one, at least three, at least five, at least ten) predictors from “patterns of non-opioid prescription use” predictors listed in Table 1. In some embodiments, the data associated with the subject comprises values for at least some (e.g., at least one, at least three, at least five, at least ten) predictors from “beneficiaries sociodemographics” predictors listed in Table 1. In some embodiments, the data associated with the subject comprises values for at least some (e.g., at least one, at least three, at least five, at least ten) predictors from “health status factors” predictors listed in Table 1. In some embodiments, the data associated with the subject comprises values for at least some (e.g., at least one, at least three, at least five, at least ten) predictors from “opioid prescriber-level” predictors listed in Table 1. In some embodiments, the data associated with the subject comprises values for at least some (e.g., at least one, at least three, at least five, at least ten) predictors from “regional-level factors” predictors listed in Table 1. In some embodiments, the data associated with the subject comprises values for at least the predictors listed in column 1 of Table 7, column 2 of Table 7, or column 3 of Table 7.

In some embodiments, the techniques include determining whether to intervene with the subject based on the output indicative of the risk of OUD and/or of the opioid overdose episode for the subject. In some embodiments, in response to determining to intervene with the subject, an intervention may include: selecting the subject for enrollment in a lock-in program, making an outreach call to the subject, referring the subject to a use disorder specialist, prescribing an opioid antagonist therapy, and/or administering an opioid antagonist therapy to the subject. In some embodiments, the opioid antagonist therapy may comprise naloxone or naltrexone. In some embodiments, an intervention with naloxone distribution may be included for a subject at high risk of an opioid overdose episode. In some embodiments, an intervention for a subject at high risk of development of OUD may comprise naloxone distribution, or initiating medications used for OUD treatment including naltrexone, and/or buprenorphine. In some embodiments, administering the opioid antagonist therapy comprises administering the therapy orally, parenterally, by inhalation spray, topically, nasally, and/or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial or infusion techniques.

Some embodiments provide for a method for using a trained machine learning model to predict risk of OUD and/or of an opioid overdose episode for a subject, the method comprising: accessing data associated with the subject, wherein the data comprises values for one or more social determinants of health; generating input features for the trained machine learning model from the data; and providing the input features as input to the trained machine learning model to obtain an output indicative of the risk of OUD and/or of the opioid overdose episode for the subject, wherein the trained machine learning model comprises a first plurality of values for a respective first plurality of parameters, the first plurality of values used by the at least one computer hardware processor to obtain the output from the input features.

In some embodiments, the values for the one or more social determinants of health include values for one or more predictors indicating: economic stability, education level, community context, child abuse history, family history of substance abuse, and/or whether the subject is in jail. In some embodiments, the data associated with the subject comprises values for one or more of the predictors listed from the “Social determinants of health” category in Table 2 below.

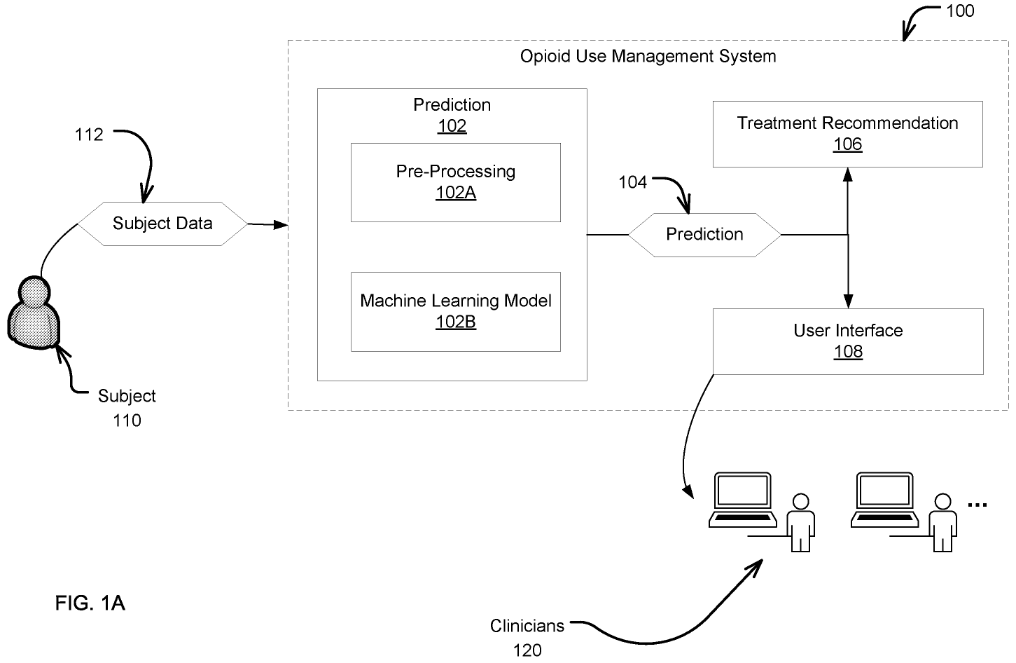

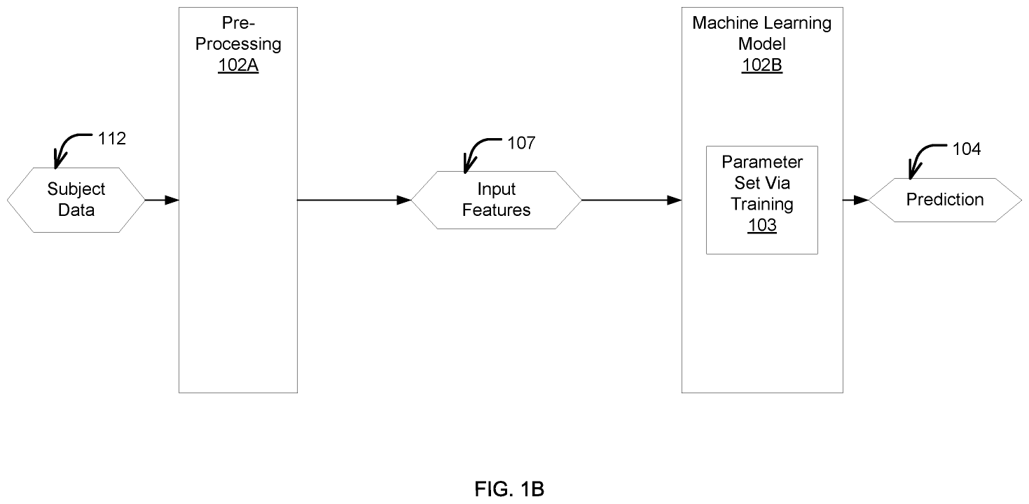

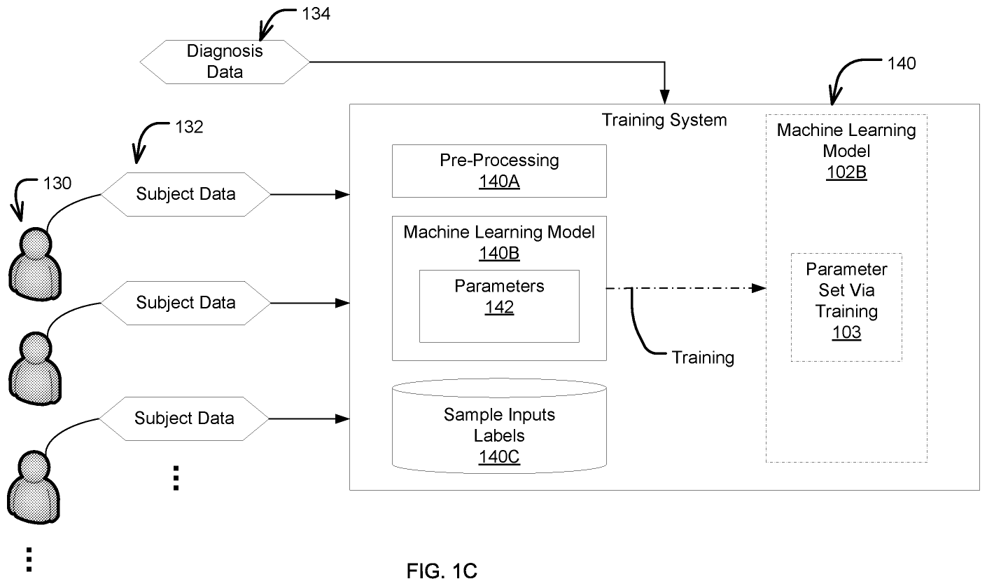

FIG. 1A is a diagram illustrating an example opioid use management system 100, according to some embodiments of the technology described herein. The system 100 obtains data 112 for a subject 110. The system 100 provides information (e.g., prediction of risk of OUD and/or an opioid overdose episode for the subject 110) to computing devices of one or more clinicians 120.

In some embodiments, the subject 110 may be a person whose opioid use may be managed using the opioid use management system 100. In some embodiments, the subject 110 may be a person who has been prescribed opioid medication. For example, the subject 110 may have been prescribed opioid medication by a care provider (e.g., by a physician, nurse, physician's assistant, etc.). Data 112 about the subject 110 may be collected (e.g., for use by the opioid use management system 100). The data 112 may include some or all of the information listed in Tables 1 and 2 below.

TABLE 1
Candidate Predictors

| Patterns of | Pattens of non- | | | Opioid | |
|---|---|---|---|---|---|
| prescription | opioid | | | prescriber- | |
| opioid | prescription | Beneficiaries | Health Status | level | Regional-level |
| use | use | sociodemographics | Factors | predictors | factors |
| Average | No. BZD fills | Age | No. outpatient visits | Prescriber's | AHRF total |
| opioid daily | No. muscle | Sex | No. ED visits | sex | health facilities |
| dose in | relaxants fills | Race | No. inpatient visits | Prescribers | predictors |
| MME | Cumulative | State of residence | History of | specialties | AHRF health |
| Cumulative | overlapping | County of | prescription opioid | Average | professions |
| MME | days of | residence | overdose | monthly | predictors |
| Cumulative | concurrent | Zip code of | History of heroin | opioid | AHRF health |
| duration for | opioid and | residence | overdose | prescribing | training |
| any opioids, | BZD use | Type of county of | Non-opioid drug | volume | programs |
| SAO, and | Cumulative | residence (metro | use disorders | Average | predictors |
| LAO | overlapping | vs. non-metro) | Other non-opioid | monthly | AHRF hospital |
| Duration of | days of | Disabled | SUD or alcohol use | opioid | expenditure, |
| longest | concurrent | eligibility | disorders | prescribing | Medicare costs, |
| continuous | opioid and | Receipt of low- | Alcohol use | dose in | VA |
| use for any | muscle | income subsidy | disorders | MME | expenditure |
| opioids, | relaxants use | | Personality | Average | AHRF |
| SAO, and | Cumulative | | disorders | monthly | inpatient |
| LAO | overlapping | | Psychoses | patients | days/discharges |
| No. fills of | days of | | Delusional | receiving | predictors |
| any opioids, | concurrent | | disorders | opioids | AHRF other |
| SAO, and | opioid, BZD | | Schizophrenia | | health services |
| LAO | and muscle | | Mood disorders | | utilization |
| No. | relaxants use | | Anxiety disorders | | predictors |
| standardized | Cumulative | | Alcohol-induced | | AHRF census- |
| 30-day | duration of | | mental disorders | | based |
| prescriptions | buprenorphine | | Drug-induced | | predictors (e.g., |
| for any | for opioid use | | mental or sleep | | medium |
| opioids, | disorder | | disorders | | household |
| SAO, and | Cumulative | | Other mental health | | income, |
| LAO | duration of | | disorders | | employment) |
| Cumulative | naltrexone | | Osteoarthritis | | AHRF health |
| duration of | No. | | Rheumatoid | | insurance |
| 30-day use | gabapentinoid | | arthritis | | status |
| of any | fills | | Back pain | | predictors |
| opioids, | Cumulative | | Neck pain | | AHRF housing |
| SAO, and | duration of | | Headache or | | statistics |
| LAO | gabapentinoid | | migraine | | Area |
| No. fills by | use | | Temporomandibular | | deprivation |
| opioid | No. | | disorder pain | | index |
| ingredient | antidepressants | | Abdominal pain or | | County-health |
| and type | fills | | hernia | | ranking |
| (e.g., any | Cumulative | | Chest pain | | predictors |
| fentanyl, | duration of | | Kidney or gall | | |
| SAO-type | antidepressant | | bladder stones | | |
| fentanyl, | use | | Menstrual or genital | | |
| LAO-type | No. average | | reproductive pain | | |
| fentanyl) | monthly non- | | Fractures, | | |
| Type of | opioid | | concussion, injuries | | |
| opioids by | prescriptions | | Fibromyalgia | | |
| Schedule | No. naltrexone | | Internal orthopedic | | |
| and | fills | | device implant/graft | | |
| SAO/LAO | | | Other pain | | |
| (e.g., SAO, | | | conditions | | |
| Schedule I | | | Surgical procedures | | |
| only) | | | (e.g., ischemic heart | | |
| No. unique | | | disease) | | |
| opioid | | | Diseases of | | |
| prescribers | | | musculoskeletal | | |
| No. unique | | | system and | | |
| pharmacies | | | connective tissues | | |
| No. early | | | Neuropathies | | |
| refills for | | | (excluding | | |
| opioids | | | alcoholic, drug, and | | |
| Cumulative | | | optic-related) | | |
| overlapping | | | Ischemic heart | | |
| days of early | | | disease | | |
| refills | | | HIV/AIDS | | |
| Use of | | | Elixhauser index | | |
| injectable | | | and individual | | |
| opioids or | | | categories | | |
| antitussive | | | | | |
| opioids | | | | | |

Abbreviations:
AHRF; Area Health Resources Files;
BZD: benzodiazepines;
LAO: long-acting opioids;
MME: morphine milligram equivalent;
No: number of;
SAO: short-acting opioids;
SUD: substance use disorders.

TABLE 2
Candidate Predictors

| Categories | Features measured | Sources |
|---|---|---|
| Opioid use patterns | Average MME; cumulative and continuous duration of opioid use overall and by | CDM/claims |
| 135,137-139,164-168 | type (e.g., short-acting); total no. opioid prescriptions (overall and by | |
| | ingredient); type of opioid; no. prescribers and pharmacies for opioid | |
| | prescriptions; no. and cumulative days of early refills; cumulative days of | |
| | concurrent use with benzodiazepines; muscle relaxants, and/or gabapentinoids; | |
| | for overdose outcome only: methadone for OUD (procedure codes: H0020 and | |
| | J1230), and buprenorphine/naltrexone fills for OUD | |

Patient-level features

| | | |
|---|---|---|
| Patient | Age; sex; race (white, black, Hispanic, American Indians, others); ethnicity; | CDM/claims |
| sociodemographics | language spoken (CDM only); type of insurance; Medicaid eligibility type | |
| | (claims only) | |
| Health status | No. outpatient, ED, and inpatient utilization; time to prior overdose events; no. | Claims/CDM |
| factors^{27,143,144,169,170} | antidepressants; no. non-opioid prescriptions; substance use disorders; mental | |
| | conditions; chronic pain conditions; use of subspecialties; Elixhauser index and | |
| | individual conditions, substance use disorder counseling, and urine drug screens | |
| | *Vital statistics; body mass index; smoking and tobacco use and duration; | CDM only |
| | medications filled in ED or inpatient settings (e.g., naloxone, naltrexone) | |
| Social | *Economic stability (e.g., “homeless living in a shelter”) | NLP (C.4.5.1) |
| determinants of | *Education (e.g., “dropped out of high school”, “limited health literacy”) | |
| health (SDoH; | *Social and community context (e.g., “avoids social situation”, “live alone”) | |
| concept example in | *Others (e.g., “childhood abuse”, “family history of substance use”, “in jail”) | |
| clinical notes)* | | |
| Behavioral | *Smoking (e.g., former history of tobacco use); alcohol abuse (e.g., “pt drank | NLP (C.4.5.1) |
| determinants of | ETOH”); marijuana use (medical or recreational); substance use and abuse | |
| health (BDoH)* | *Suicidal ideation; suicidal attempts | |
| Pain-related | *Pain severity (e.g., validated numeric rating scale); Pain and mental health | NLP (C.4.5.1) |
| determinants* | conditions (e.g., insomnia), pain management methods; obtaining pain killers | |
| | from others | |
| Lab/imaging | Lab or imaging orders (CDM/claims) | CDM/claims |
| | *Lab results (CDM only) such as urine drug results; HbAIC; lipid panels; x-rays | |

Provider-, policy, system and regional-level features

| | | |
|---|---|---|
| Provider factors | Opioid prescriber's sex; specialty; monthly opioid prescribing volume and dose; | CDM or claims |
| Policy, system and | *Year of opioid-related policies implemented in FL | FL state, |
| regional factors | County- and zip code-level predictors | AHRF,CHR&R |

Abbreviations:
CDM: common data model;
MME: morphine milligram equivalent;
NLP: natural language processing;
no.: number of

In some embodiments, subject data 112 may be collected at multiple points over a period of time. For example, subject data 112 may be collected at regular intervals (e.g., every day, every week, every month, every appointment with a care provider). In some embodiments, data collection of the subject data 112 may initiate when the subject 110 is first prescribed an opioid. In some embodiments, data collection of the subject data 112 may initiate prior to the subject 110 receiving a first opioid prescription. For example, the data collection of the subject data 112 may initiate a period of time (e.g., 1, 2, 3, 4, 5, or 6 months) prior to the first opioid prescription.

As shown in FIG. 1A, opioid use management system 100 includes a prediction module 102, a treatment recommendation module 106, and a user interface module 108. In some embodiments, each of the modules 102, 106, 108 may include a set of processor-executable instructions that, when executed by the opioid use management system 100, cause the opioid user management system 100 to perform described fimctions of the module. In some embodiments, the opioid user management system 100 may include one or more modules instead of or in addition to the modules 102, 106, 108 shown in FIG. 1A.

As shown in FIG. 1A, the opioid use management system 100 may be configured to obtain the subject data 112. In some embodiments, the opioid use management system 100 may be configured to obtain the subject data 112 from another system. For example, the opioid use management system 100 may obtain the subject data 112 from an electronic health record (EHR) system (e.g., used by a medical care facility at which the subject 110 is receiving care). In some embodiments, the system 100 may be configured to obtain the subject data 112 through a communication network (e.g., the Internet). The opioid use management system 100 may be configured to obtain the data 112 from the EHR system through an application program interface (API). In some embodiments, the opioid use management system 100 may be configured to obtain the subject data 112 by receiving one or more files including at least some of the subject data 112. For example, the opioid use management system 100 may receive one or more data files (e.g., CSV, JSON, or any other suitable file type and/or format) including the at least some of the subject data 112. In some embodiments, the opioid use management system 100 may be configured to obtain the subject data 112 through a graphical user interface (GUI). For example, the opioid use manauement system 100 may be configured to obtain user input specifying at least some of the subject data 112 through the GUI.

In some embodiments, the subject data 112 may be updated (e.g., periodically). For example, the subject data 112 may be updated by appending values of predictors in the subject data 112 collected at different points in time. In another example, the subject data 112 may be updated by replacing a previous value of a predictor with a newly obtained more recent value of the predictor. In yet another example, the subject data 112 may be updated by accumulating values of a predictor (e.g., by summing, appending, or averauinu) collected at different points in time. In some embodiments, the subject data 112 may be updated periodically. For example, the subject data 112 may be updated every day, week, month, 3 months, 6 months, year, or other suitable frequency.

In some embodiments, the prediction module 102 of the opioid use management system 100 may be configured to use the subject data 112 to generate a prediction 104 for the subject 110. The prediction 104 may be a predicted risk of OUD and/or an opioid overdose episode for the subject 110 during a period of time. As shown in FIG. 1A, the prediction module 102 includes a pre-processing module 102A and a machine learning model 102B.

FIG. 1B is a diagram illustrating example operation of the prediction module 102 of the opioid use management system 100 of FIG. 1A, according to some embodiments of the technology described herein. As shown in FIG. 1B, the pre-processing module 102A: (1) obtains the subject data 112; and (2) generates input features 107 using the subject data 112. In some embodiments, the pre-processing module 102A may be configured to one-hot encode one or more categorical predictors (e.g., race, state of residence, disorders) in the subject data 112 to uenerate one or more input features. In some embodiments, the pre-processing module 102A may be configured to standardize one or more numerical predictors (e.g., age, number of files of an opioid, number of opioid prescriptions, duration of longest opioid use) of the subject data 112. For example, the pre-processing module 102A may standardize the numerical predictor by normalizing the numerical predictor. In some embodiments, the pre-processing module 102A may be configured to generate an input feature by combining values of one or more predictors of the subject data 112. For example, the pre-processing module 102A may generate an input feature to be a weighted combination of one or more numerical predictors (e.g., number of antidepressant fills and/or number of opioid fills). In some embodiments, the pre-processing module 102A may be configured to impute missing predictor values. For example, the pre-processing module may impute a missing value with a mean, mode, maximum, or minimum value of the predictor.

In some embodiments, the pre-processing module 102A may be configured to select a subset of the subject data 112 and use the selected subset of data to generate the input features 107. For example, the pre-processing module 102A may select 5, 10, 15, 20, 25, 30, 35, 40, 45, 50, 60, 70 80, 90, 100, 150, or 200 of the predictors shown in Table 1 and/or Table 2 and use the selected predictors to generate the input features 107. In some embodiments, the pre-processing module 102A may be configured to generate the input features 107 by reducing dimensions of the subject data 112 to obtain a representation of the subject data 112 with lower dimensionality. In some embodiments, the pre-processing module 102 may be configured to generate the input features 107 by combining multiple data points collected over a period of time. For example, the pre-processing module 102 may generate an input feature by determining a mean, mode, maximum, minimum, or combination of values of a predictor in the subject data 112 collected over a period of time.

In some embodiments, the pre-processing module 102A may be configured to determine values for predictors of subject data 112 that do not have a value. For example, some of the predictors in the subject data 112 may not have values (e.g., due to information not being available). The pre-processing module 102A may be configured to impute a value for the input features. In some embodiments, the pre-processing module 102A may be configured to impute a value for a continuous predictor with a mean, median, or mode of the predictor (e.g., determined from a training data set). In some embodiments, the pre-processing module 102A may be configured to impute a value for a categorical predictor with the most frequent category (e.g., determined from a training data set).

As shown in FIG. 1B, the prediction module 102 provides the input feature 107 generated by the pre-processing module 102A as input to the machine learning model 102B to obtain the prediction 104. The machine learning model 102B includes a parameter set 103 obtaining via training. For example, the machine learning model 102B may be a deep neural network (DNN) in which the parameter set 103 includes a number of trained weights of layers of the DNN. In another example, the machine learning model 102B may be a logistic regression model with coefficients obtained via training. In another example, the machine learning model 102B may be a random forest model or a gradient boosting machine model. In another example, the machine learning model 102B may include a support vector machine (SVM), a clustering model, or any other suitable type of machine learning model 102B.

In some embodiments, the prediction model 102 may be configured to use the parameter set 103 obtained via training to process the input features 107. For example, the prediction module 102 may process the input features 107 by using the input features 107 as values of inputs to a function (e.g., a logistic regression function). In another example, the input features 107 may be stored in a vector or matrix, and the parameter set 103 may be stored in a matrix or vector. The prediction module 102 may be configured to apply the matrix of parameter set values 103 by those of the input features 107 to obtain the prediction 104 (e.g., by performing matrix multiplication or another suitable operation). For example, the machine learning model 102B may be a neural network with multiple layers, where the weights of each layer are stored in a matrix or vector.

The prediction 104 may be an output of the machine learning model 102B. In some embodiments, the prediction 104 may be a classification. In some embodiments, the prediction 104 may be a classification of a risk of OUD and/or of an opioid overdose episode. For example, the prediction 104 may be a classification into one of low, medium, and high risk levels. In another example, the prediction 104 may be a classification of the subject 110 into one of multiple opioid-benzodiazepine dosage patterns. In some embodiments, the prediction 104 may be a numerical value (e.g., a likelihood or score) indicating a risk of the subject 110 developing OUD and/or having an episode of opioid overdose. For example, the prediction 104 may be a value between 0 and 1 in which a value of 0 indicates the lowest level of predicted risk and a value of 1 indicates the highest level of predicted risk. In another example, the prediction 104 may be a predicted probability of the subject 110 developing OUD and/or having an opioid overdose episode.

In some embodiments, the prediction 104 may be a predicted risk of OUD and/or of an opioid overdose episode in a period of time. In some embodiments, the period of time may be a period of time after a period of collecting subject data 112. For example, the period of time may be a period of 1 week, 1 month, 2 months, 3 months, 4 months, 5 months, 6 months, 7 months, 8 months, 9 months, 10 months, 11 months, or 1 year after a period of collecting subject data 112. The prediction 104 may indicate a risk of OUD and/or of an opioid overdose episode in the period of time. The input features 107 may be generated from subject data 112 collected prior to the period of time. For example, the input features 107 may be generated from subject data 112 collected for a period of time (e.g., a previous 3 months) and/or all accumulated subject data 112 collected up to a certain point.

In some embodiments, the prediction 104 may be a classification of the subject 110 based on an output (e.g., probability or score) of the machine learning model 102B. In some embodiments, the prediction 104 may be a binary classification of whether the subject 110 is predicted to develop OUD and/or have an episode of opioid overdose. The prediction module 102 may be configured to determine the classification by comparing the output of the machine learning model 102B to a threshold value. The prediction module 102 may be configured to predict that the subject 110 will develop OUD and/or have an episode of opioid overdose if the output is greater than the threshold value, and to predict that the subject 110 will not develop OUD and/or have an episode of opioid overdose when the output is less than the threshold value. In some embodiments, the threshold value may be configurable. For example, the threshold value may be modified to adjust sensitivity of the prediction module 102 in predicting a subject to develop OUD and/or have an episode of opioid overdose. A lower threshold value may increase the likelihood that a subject is predicted to develop OUD and/or have an episode of opioid overdose. A higher threshold value may decrease the likelihood that a subject is predicted to develop OUD and/or have an episode of opioid overdose.

Returning to FIG. 1A, the treatment recommendation module 106 may be configured to use the prediction 104 to determine a treatment recommendation for a subject. In some embodiments, the treatment recommendation may be an adjustment in medication for the subject 110. For example, the treatment recommendation may include a suggested change in dosage, replacement of opioid medication, or other adjustment in medication. In some embodiments, the treatment recommendation module 106 may be configured to determine a treatment recommendation based on a level of predicted risk for the subject 110. For example, the treatment recommendation module 106 may be configured to determine a first recommendation for the subject 110 when the prediction 104 is a high risk level, a second recommendation when the prediction 104 is a medium risk level, and a third recommendation when the prediction 104 is a low risk level. In some embodiments, the treatment recommendation module 106 may be configured to determine a treatment recommendation based on the input features 107 and/or the subject data 112. For example, the treatment recommendation module 106 may: (1) identify input predictors of the subject data 112 that contributed most to the prediction 104; and (2) determine the treatment recommendation based on the identified predictors of the subject data 112.

The user interface module 108 may be configured to generate a graphic user interface (GUI) displaying a prediction 104 and/or a treatment recommendation determined by the treatment recommendation module 106. The user interface module 108 may be configured to generate the GUI for display on one or more computing devices (e.g., used by one or more clinicians 120). The GUI may allow clinician(s) 120 to access information about the subject 110. The user interface module 108 may be configured to generate a GUI displaying information about medication being uiven to the subject 110. For example, the GUI may display a type of medication, and dosage thereof. The GUI may display a risk of OUD and/or of an opioid overdose episode for the subject 110 based on the prediction 104 generated by the prediction module 102. In some embodiments, the user interface module 108 may be configured to generate a GUI displaying a risk level (e.g., low, medium, or high) for the subject 110 and/or a treatment recommendation (e.g., an adjustment in medication). In some embodiments, the user interface module may be configured to generate a GUI displaying risk of OUD and/or of an opioid overdose episode for the subject 110. The GUI may display a predicted risk for various periods of time. For example, the GUI may display a predicted risk of developing OUD and/or having an opioid overdose episode in the next 3 months, 6 months, and 12 months. In some embodiments, the user interface module 108 may be configured to generate a GUI displaying information about a strength of predictors in generating the prediction 104.

FIG. 1C is a diagram illustrating an example training system 140 for training a machine learning model 140B, according to some embodiments of the technology described herein. As indicated by the dotted line labeled “Training” in FIG. 1C, the training system 140 trains the machine learning model 140B to obtain the machine learning model 102B with parameter set 103 described with reference to FIGS. 1A-B.

As shown in FIG. 1C, the training system 140 obtains subject data 132 from multiple different subjects 130. The subject data 132 may include values of various different predictors such as those shown in Table 1 and Table 2 above. The training system 140 receives diagnosis data 134 for the subjects. The diagnosis data 134 may be information indicating diagnosis of OUD for the subjects 130 and/or occurrence of opioid overdose for the subjects 130. In some embodiments, the diagnosis data 134 may be diagnosis codes for the subjects 130 indicating diagnosis of opioid overdose and/or opioid overdose. Table 3 below shows examples of diagnosis codes that may correspond to medical diagnosis of opioid overdose and OUD. Table 4 below shows diagnosis codes associated with other medical diagnoses that may also indicate diagnosis of opioid overdose and/or OUD in a subject.

TABLE 3
Opioid Overdose and OUD Diagnosis Codes

| Conditions | ICD-9 Codes | ICD-10 Codes |
|---|---|---|
| Opioid Overdose | 965.00, 965.01, 965.02, 965.09, | T40.0X1A, T40 [symbol] |
| | E850.0, E850.1, E850.2, E935.0, | T40.0X4A, T40 [symbol] |
| | E935.1, E935.2 | T40.1X3A, T40 [symbol] |
| | | T40.2X2A, T40 [symbol] |
| | | T40.3X1A, T40 [symbol] |
| | | T40.3X4A, T40 [symbol] |
| | | T40.4X3A, T40 [symbol] |
| | | T40.602A, T40 [symbol] |
| | | T40.691A, T40 [symbol] |
| | | T40.694A |
| Incident Opioid | 304.0X, 304.7X, 305.5X | F11.1X and F11 [symbol] |
| Use Disorder | | (opioid-related [symbol] |
| | | and F11.21 (opi [symbol] |
| | | remission) |

[symbol]

indicates data missing or illegible when filed

TABLE 4
Other Codes for Opioid Overdose and OUD

| | | |
|---|---|---|
| ICD | ICD | |
| type | code | ICD codes description |

Other drug/substance-related overdose or substance use disorders

| | | |
|---|---|---|
| ICD-9 | 965* | Poisoning by analgesics antipyretics and anti-rheumatics |
| ICD-9 | 966 | Poisoning by anticonvulsants and anti-parkinsonism drugs |
| ICD-9 | 967 | Poisoning by sedatives and hypnotics |
| ICD-9 | 968 | Poisoning by other central nervous system depressants and anesthetics |
| ICD-9 | 969 | Poisoning by psychotropic agents |
| ICD-9 | 970 | Poisoning by central nervous system stimulants |
| ICD-9 | 971 | Poisoning by drugs primarily affecting the autonomic nervous system |
| ICD-9 | 972 | Poisoning by agents primarily affecting the cardiovascular system |
| ICD-9 | 973 | Poisoning by agents primarily affecting the gastrointestinal system |
| ICD-9 | 975 | Poisoning by agents primarily acting on the smooth and skeletal muscles |
| | | and respiratory system |
| ICD-9 | 977 | Poisoning by other and unspecified drugs and medicinal substances |
| ICD-9 | 980 | Toxic effect of alcohol |
| ICD-9 | 989 | Toxic effect of other substances chiefly nonmedicinal as to source |
| ICD-9 | 303 | Alcohol dependence syndrome |
| ICD-9 | 304 | Drug dependence |
| ICD-9 | 305 | Nondependent abuse of drugs |
| ICD-10 | F10 | Alcohol related disorders |
| ICD-10 | F11 | Opioid related disorders |
| ICD-10 | F12 | Cannabis related disorders |
| ICD-10 | F13 | Sedative, hypnotic, or anxiolytic related disorders |
| ICD-10 | F14 | Cocaine related disorders |
| ICD-10 | F15 | Other stimulant related disorders |
| ICD-10 | F16 | Hallucinogen related disorders |
| ICD-10 | F17 | Nicotine dependence |
| ICD-10 | F18 | Inhalant related disorders |
| ICD-10 | F19 | Other psychoactive substance related disorders |
| ICD-10 | T39 | Poisoning by, adverse effect of and underdosing of nonopioid |
| | | analgesics, antipyretics and antirheumatics |
| ICD-10 | T40 | Poisoning by, adverse effect of and underdosing of narcotics and |
| | | psychodysleptics [hallucinogens] |
| ICD-10 | T41 | Poisoning by, adverse effect of and underdosing of anesthetics and therapeutic gases |
| ICD-10 | T42 | Poisoning by, adverse effect of and underdosing of antiepileptic, |
| | | sedative-hypnotic and antiparkinsonism drugs |
| ICD-10 | T43 | Poisoning by, adverse effect of and underdosing of psychotropic drugs, |
| | | not elsewhere classified |
| ICD-10 | T48 | Poisoning by, adverse effect of and underdosing of agents primarily |
| | | acting on smooth and skeletal muscles and the respiratory system |
| ICD-10 | T51 | Toxic effect of alcohol |
| ICD-10 | T65 | Toxic effect of other and unspecified substances |

The training system 140 may be configured to use the diagnosis codes shown in Tables 3 and 4 to identify subjects 130 diagnosed with OUD and/or opioid overdose. The training system 140 may be configured to use the identified codes to generate labels for the subject data 132. For example, the system may determine that the diagnosis data 134 indicates an opioid overdose diagnosis code for a subject. The system may label the data associated with the subject as having a risk of an opioid overdose episode. In another example, the system may determine that the diagnosis data 134 indicates an OUD code for a subject. The system may label the data associated with the subject as one having a risk of OUD.

As shown in FIG. 1C, the training system 140 includes a pre-processing module 140A, a machine learning model 140B, and a data store 140C. The pre-processing module 140A may be configured to process the subject data 132 to generate sets of input features (e.g., for respective ones of the subjects 130). The pre-processing module 140A may be configured to generate the sets of input features as performed by pre-processing module 102A described herein with reference to FIGS. 1A-1B. In some embodiments, the pre-processing module 140A may be configured to generate labels for the sets of input features using the diagnosis data 134. For example, the pre-processing module 140A may label sets of input features as OUD positive, OUD negative, opioid overdose positive, and opioid overdose negative according to the diagnosis data 134. The pre-processing module 140A may be configured to store the generated sets of input features (also referred to as “sample inputs”) and corresponding labels in the data store 140C. The sample inputs and the labels may be used as training data.

The machine learning model 140B may be any suitable machine learning model. In some embodiments, the machine learning model 140B may be a neural network. For example, the machine learning model 140B may be a deep neural network (DNN), convolutional neural network (CNN), recurrent neural network (RNN), or other suitable type of neural network. In some embodiments, the machine learning model 140B may be a logistic regression model. For example, the machine learning model 140B may be a binary logistic regression model, a multinomial logistic regression model, an ordinal logistic regression model, or other suitable type of logistic regression model. In some embodiments, the machine learning model 140B may be a random forests (RF) model or a gradient boosting machine (GBM).

In some embodiments, the training system 140 may be configured to train the machine learning model 140B using the training data (e.g., sample inputs and labels stored in data store 140C). The machine learning model 140B includes parameters 142. The training system 140 may be configured to train the parameters by applying a training algorithm to the training data to obtain the machine learning model 102B with parameter set 103 obtained via the training. In some embodiments, the training system 140 may be configured to perform a supervised learning technique using the training data to train the machine learning model 140B. For example, the training system 140 may perform stochastic gradient descent using the training data to train the machine learning model 140B (e.g., a neural network) to obtain the machine learning model 102B. In another example, the training system 140 may perform gradient boosting to train the machine learning model 140B to obtain the machine learning model 102B. In another example, the training system 140 may perform Elastic net regularization to train the machine learning model 140B to obtain the machine learning model 102B. In some embodiments, the training system 140 may be configured to perform an unsupervised learning technique using the training data. For example, the training system 140 may perform a k-nearest neighbor (KNN) algorithm using the training data to generate a clustering model. In some embodiments, the training system 140 may be configured to perform a semi-supervised learning technique.

In some embodiments, the data store 140C may include one or more storage devices storing data in one or more formats. In some embodiments, the data store 140C may include one or multiple storage devices storing data in one or more formats of any suitable type. For example, the storage device(s) part of a data store may store data using one or more database tables, spreadsheet files, flat text files, and/or files in any other suitable format (e.g., a native format of a mainframe). The storage device(s) may be of any suitable type and may include one or more servers, one or more database systems, one or more portable storage devices, one or more non-volatile storage devices, one or more volatile storage devices, and/or any other device(s) configured to store data electronically. In embodiments where a data store includes multiple storage devices, the storage devices may be co-located in one physical location (e.g., in one building) or distributed across multiple physical locations (e.g., in multiple buildings, in different cities, states, or countries). The storage devices may be configured to communicate with one another using one or more networks of any suitable type, as aspects of the technology described herein are not limited in this respect.

As shown in FIG. 1C, the data store 140C stores sample inputs and corresponding labels. The training system 140 may be configured to update the sample inputs and labels. For example, as subject data from additional subjects is obtained and/or as subject data 132 previously obtained is updated, the training system 140 may update the sample inputs and labels (e.g., by generating new sets of input features and corresponding labels). The training system 140 may be configured to update a previously trained machine learning model. For example, the machine learning model 140B may be a previously trained machine learning model. The training system 140 may retrain the previously trained machine learning model by training using updated training data (e.g., updated sample inputs and/or labels).

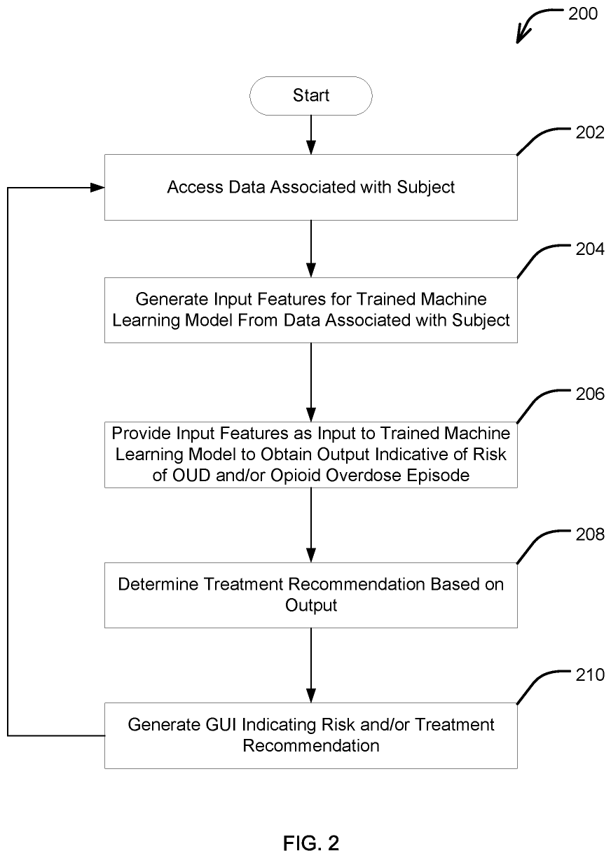

FIG. 2 is a diagram illustrating an example process 200 of using a trained machine learning model to predict risk of OUD and/or of an opioid overdose episode for a subject, according to some embodiments of the technology described herein. Process 200 may be performed by any suitable computing device. In some embodiments, process 200 may be performed by opioid use management system 100 described herein with reference to FIGS. 1A-1B.

Process 200 begins at block 202, where the system accesses data associated with a subject. For example, the data may be subject data 112 described herein with reference to FIG. 1A. The data associated with the subject may include values of various predictors. In some embodiments, the data may include values of at least some of the predictors shown in Table 1 and/or Table 2.

The system may be configured to access data associated with the subject in any suitable way. In some embodiments, the system may be configured to access the data associated with the subject from another system. For example, the system may access the data from an EHR system storing information about the subject. The system may be configured to receive, through a communication network (e.g., the Internet) the data associated with the subject. In some embodiments, the system may be configured to receive data associated with the subject in one or more documents. For example, one or more data files (e.g., CSV, JSON, or any other suitable file type and/or format) storing data may be uploaded to the system. In some embodiments, the system may be configured to receive data through a graphical user interface (GUI) generated by the system. For example, the system may generate a GUI that allows a user (e.g., a clinician) to input data associated with the subject.

Next, process 200 proceeds to block 204, where the system generates input features for a trained machine learning model (e.g., machine learning model 102B) from the data associated with the subject. In some embodiments, the system may be configured to generate input features for the trained machine learning model by transforming one or more values of predictors from the data associated with the subject. For example, the system may one-hot encode predictors with multiple possible categories. In another example, the system may standardize continuous predictors (e.g., by normalizing between 0 and 1). In some embodiments, the system may be configured to impute values for one or more features. For example, the data may not include for one or more predictors. The system may impute value(s) for features generated from the predictor(s) (e.g., by imputing a median value for continuous predictor(s) and/or the most frequent category for a categorical predictor). In some embodiments, the system may be configured to combine values of multiple predictors in the data to generate a respective input feature. For example, the system may determine a linear combination of the multiple predictors to generate the respective input feature. The system may be configured to generate the input features in any suitable form. In some embodiments, the system may be configured to generate the input features as a vector, where each entry stores a value of a respective feature. For example, the

In some embodiments, the system may be configured to generate input features by selecting a set of predictors from a set of candidate predictors. During training, a set of predictors (e.g., selected from those shown in Table 1 and/or Table 2) may be identified for use in predicting OUD and/or opioid overdose. For example, a set of 5, 10, 15, 20, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 90, 100, 125, or 150 predictors may be identified for prediction. In another example, the set of predictors may include 1-5, 5-10, 10-15, 10-20, 10-25, 10-30, 10-50, 25-50, 50-75, 50-100, 100-125, 100-150, or 125-150 predictors. The system may be configured to: (1) determine values for the set of predictors from the data associated with the subject; and (2) generate input features using the values for the set of predictors. A machine learning model that uses a fewer number of predictors may require fewer parameters, and thus may be trained and used for inference more efficiently by a computing device.

Next, process 200 proceeds to block 206, where the system provides the input features as input to a trained machine learning model to obtain output indicative of a risk that the subject will develop OUD and/or have an opioid overdose episode. The output may be the prediction 104 described herein with reference to FIGS. 1A-1B. In some embodiments, the system may be configured to provide a vector storing the input features as input to a machine learning model (e.g., neural network, logistic regression model, RF model, GBM model) to obtain an output. In some embodiments, the output may indicate a classification for the subject. For example, the output may indicate a binary classification of the subject as being predicted to have a risk of OUD or to not have a risk of OUD in a period of time (e.g., in three months). In another example, the output may indicate a binary classification of the subject as being predicted to have an opioid overdose episode or to not have an opioid overdose episode in a period of time (e.g., in three months). In some embodiments, the output may indicate a classification into a risk level for developing OUD and/or having an opioid overdose episode. For example, the output may indicate a classification of low risk, medium risk, or high risk of OUD and/or an opioid overdose episode in a period of time. In some embodiments, the output may indicate a likelihood (e.g., a probability) of OUD and/or an opioid overdose episode in a period of time. For example, the output may be a score or a probability of OUD and/or an opioid overdose episode.

Next, process 200 proceeds to block 208, where the system determines a treatment recommendation based on the output. In some embodiments, the system may be configured to determine a treatment recommendation based on a classification indicated by the output. When the classification is a prediction that the subject will develop OUD and/or have an opioid overdose episode, the system may determine a modification in medication. When the classification is a prediction that the subject will not develop OUD and/or have an opioid overdose episode, the system may determine that no modification is needed. In some embodiments, the system may be configured to determine a treatment recommendation based on a risk level indicated by the output (e.g., a likelihood or a classification into one of multiple risk levels). The system may be configured to select from a set of possible treatment recommendations according to the risk level. In some embodiments, the system may be configured to determine a treatment recommendation based on the output in conjunction with other information about the subject (e.g., obtained at block 202). For example, the system may determine a treatment recommendation based on the output and a medication history, age, dosage, or other information about the subject.

Next, process 200 proceeds to block 210, where the system generates a GUI indicating the risk and/or a determined treatment recommendation. The system may be configured to generate a GUI indicating a risk as determined by the output (e.g., classification or likelihood) obtained using the machine learning model. As an example, the GUI may indicate that the subject is low risk, medium risk, or high risk based on the classification. In some embodiments, the system may be configured to indicate a risk over one or more time periods. For example, the system may indicate risk of developing OUD and/or having an opioid overdose over 3 months, 6 months, and/or 1 year. In some embodiments, the system may be configured to uenerate a GUI indicating factors that contributed to a prediction. For example, the GUI may indicate particular diagnosis identified from the data associated with the subject that contributed to a predicted risk. In some embodiments, the system may be configured to generate a GUI listing one or more treatment recommendations determined by the system at block 208. In some embodiments, the system may be configured to generate a GUI indicating the risk in conjunction with other information about the subject (e.g., medication, dosage of medication, care provider, or other information).

In some embodiments, the system may be configured to determine a treatment recommendation for the subject. For example, the system may determine an intervention such as an adjustment in medication (e.g., opioid and/or other medication), enrolling the subject into a lock-in program, dispensing medication, and/or administering opioid antagonist theory. In another example, the system may determine an evidence-based intervention for the subject (e.g., naloxone distribution, a structure of visits to healthcare providers, syringe service programs, or other evidence-based intervention). In some embodiments, the system may determine an intervention for a threat of an opioid overdose episode for the subject to be prescribing of naloxone. In some embodiments, the system may determine an intervention for a risk of OUD to be prescribing naloxone, and/or initiating medications used for OUD treatment including naltrexone and/or buprenorphine.

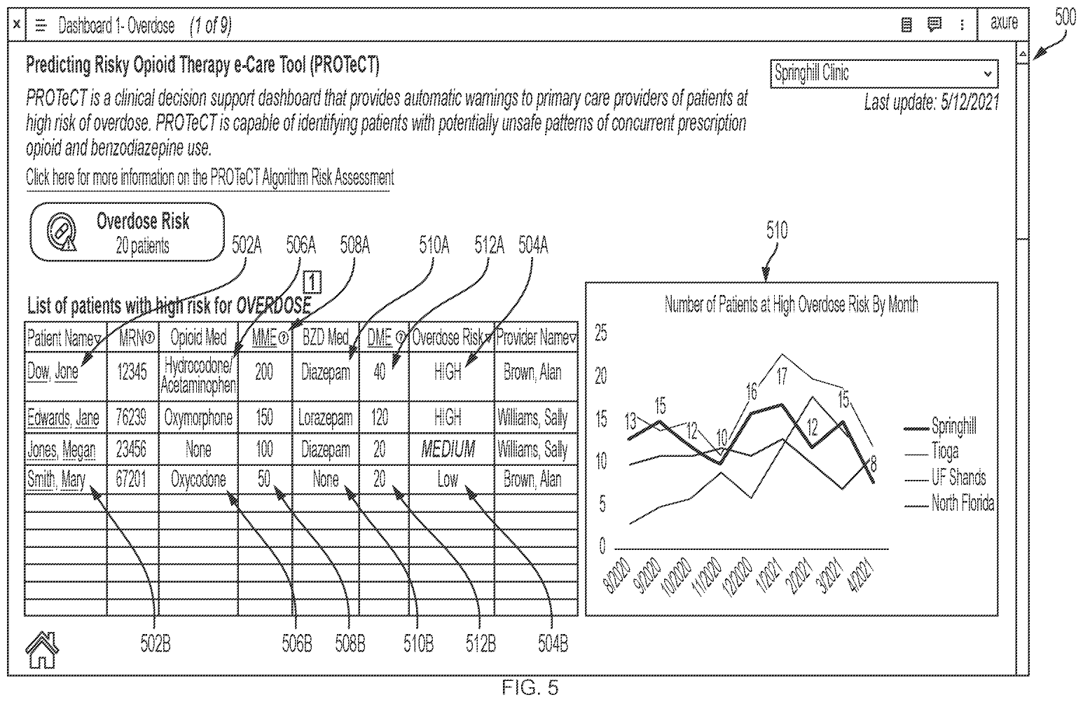

FIG. 5 is an example graphical user interface (GUI) 500 showing a prediction of risk of an opioid overdose episode for a set of subjects, according to some embodiments of the technology described herein. The GUI 500 shows a predicted risk of an opioid overdose episode (e.g., determined using an output of the machine learning model) for multiple subjects. As shown in FIG. 5, the subject “Doe, Jone” 502A is indicated to have a “HIGH” risk of an opioid overdose episode 504A. The subject “Smith, Mary” 502B is indicated to have a “LOW” risk of an opioid overdose risk episode 504B. The GUI displays other information for each of the subjects. For example, as shown in FIG. 5, the GUI 500 indicates: (1) an opioid medication “Hydrocodone/Acetaminophen” 506A for “Doe, Jone” 502A and a corresponding dosage 508A of the opioid medication; and (2) an opioid medication “Oxycodone” 506B for “Smith, Mary” 502B and a corresponding dosage 508B. The GUI 500 further displays cumulative statistics about multiple subjects. For example, as shown in FIG. 5, the GUI 500 displays a plot 510 of number of patients of a medical facility with a high risk of opioid overdose over a period of time.

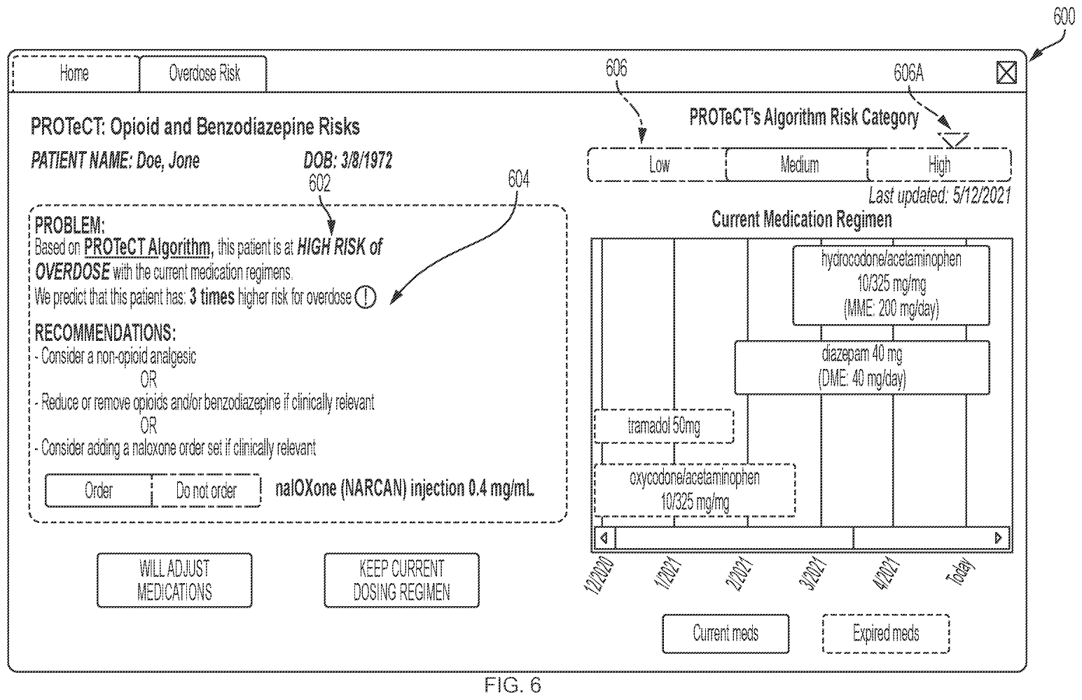

FIG. 6 is an example GUI 600 showing a prediction of risk of an opioid overdose episode for a subject and recommended treatments, according to some embodiments of the technology described herein. For example, the GUI 600 may be accessed from the GUI 500 by selecting the subject “Doe, Jone” 502A. As shown in FIG. 6, the GUI 600 indicates a risk 602 of an opioid overdose event for the subject. The GUI 600 provides a level of risk by indicating that the subject is three times more likely to overdose (e.g., relative to a median subject). The GUI 600 further indicates treatment recommendations 604 (e.g., determined at block 208) for the subject. The GUI 600 includes a graphical element 606 depicting risk of the subject of having an opioid overdose. In the example of FIG. 6, the GUI 600 indicates that the subject has a high risk 606A.

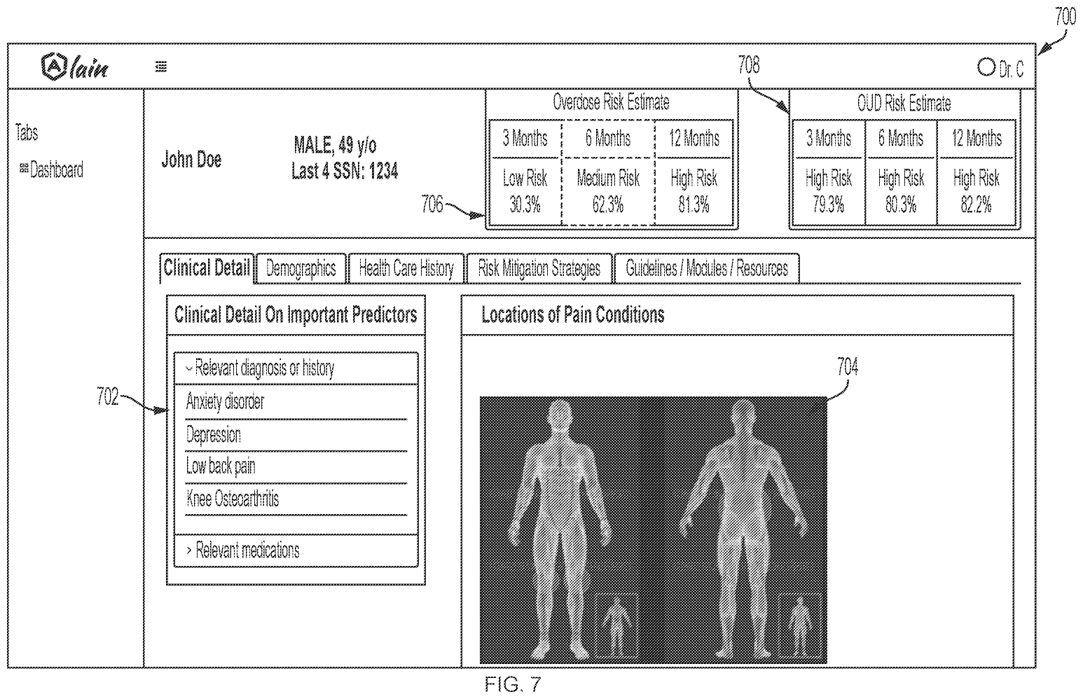

FIG. 7 is an example GUI 700 showing a prediction of risk of opioid overdose and OUD for a subject, according to some embodiments of the technology described herein. For example, the GUI 700 may be accessed from the GUI 500 by selecting a subject. The GUI 700 indicates diagnosis information 702 that contributed to the predicted risk of overdose and OUD. In the example of FIG. 7, the system determined that the subject having anxiety disorder, depression, lower back pain, and knee osteoarthritis were significant contributors to the prediction of risk of an opioid overdose event 706 and OUD 708. The GUI 700 indicates a prediction 706 of risk of an opioid overdose episode for various time periods including 3 months, 6 months, and 12 months from the date of prediction. The GUI 700 indicates a prediction 708 of risk of OUD for various time periods including 3 months, 6 months, and 12 months from the date of the prediction. In addition, the GUI 700 includes a visualization 704 of pain conditions that the subject may have.

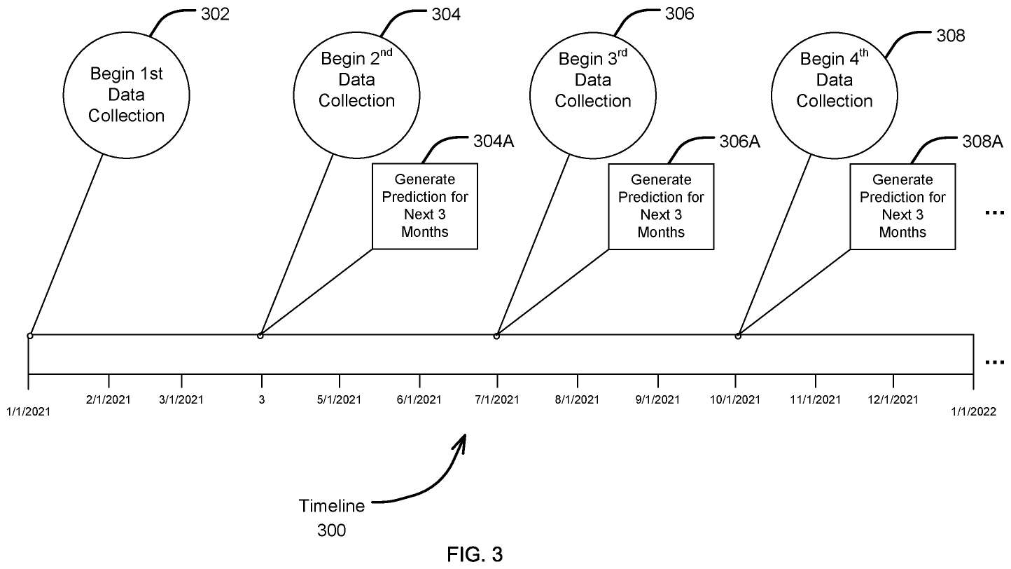

FIG. 3 is a diagram illustrating an example timeline 300 of operation of an opioid use management system, according to some embodiments of the technology described herein. The timeline 300 may be a timeline according to which opioid use management system 100 described herein with reference to FIGS. 1A-1B may operate. As shown in FIG. 3. the timeline 300 begins at 302, where the system begins collecting data about a subject. The system 100 may collect values of predictors described herein. Next, at 304, the system begins a second data collection. The system 100 further generates a prediction 304A of whether the subject will develop OUD and/or have an opioid overdose episode in the next 3 months. Next, at 306, the system begins a third data collection, and generates a prediction 306A of whether the subject will develop OUD and/or have an opioid overdose in the next three months. In some embodiments, the system may be configured to use data accumulated since the first data collection 302. In some embodiments, the system may be configured to use data collected from only the second data collection 304. Next, at 308, the system begins a fourth data collection 308, and generates a prediction 308A of whether the subject will develop OUD and/or have an opioid overdose in the next 3 months. In some embodiments, the system may be configured to use data accumulated since the first data collection 302. In some embodiments, the system may be configured to use data collected from only the fourth data collection 308. In this manner, the opioid use management system may continuously monitor a subject and generate predictions based on updated information about the subject.

It should be appreciated that the time period lengths and periods of prediction shown in FIG. 3 are exemplary. Some embodiments may perform data collection at different time intervals described herein. Some embodiments may perform predictions for different time periods described herein. For example, some embodiments may perform predictions for time periods of 1 month, 2 months, 4 months, 5 months, 6 months, 7 months, 8 months, 9 months, 10 months, 11 months, 1 year, or 2 years.

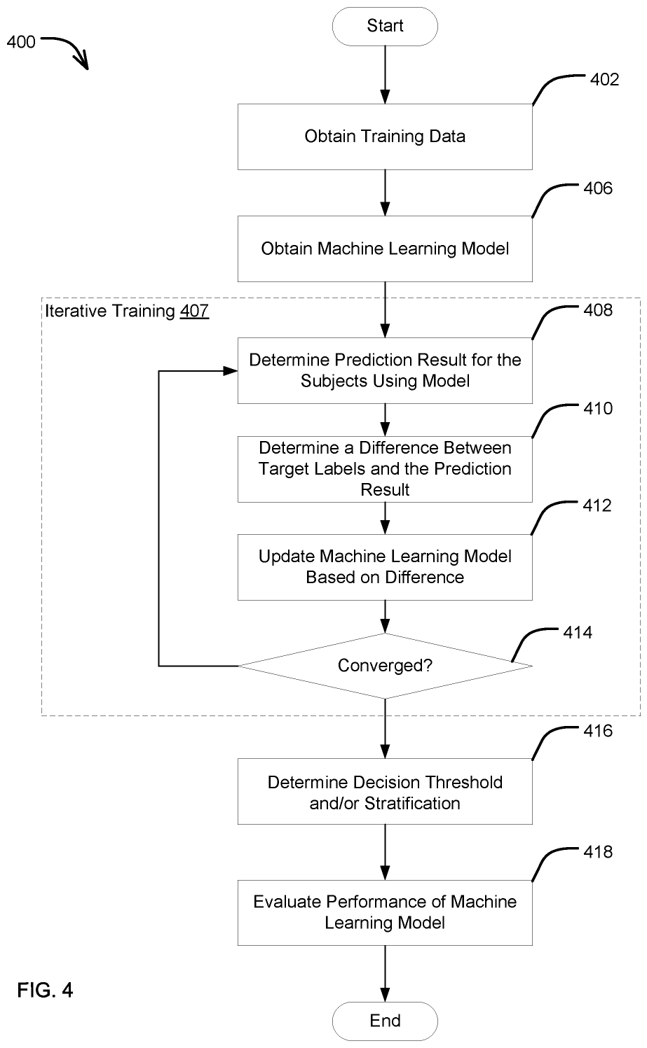

FIG. 4 is a diagram illustrating an example process 400 of training a machine learning model to predict risk of OUD and/or of an opioid overdose episode, according to some embodiments of the technology described herein. Process 400 may be performed by any suitable computing device. For example, process 400 may be performed by training system 140 described herein with reference to FIG. 1C. Process 400 may be performed to learn parameters of any suitable machine learning model. For example, process 400 may be performed to learn parameters of a neural network, a random forests model, and/or a logistic regression model.

Process 400 begins at block 402, where the system obtains training data. The system may be configured to obtain training data by obtaining predictors for multiple different subjects. The system may be configured to generate input features for each of the different subjects. For example, the system may be configured to generate an input feature for each subject as described at block 204 of process 200. In some embodiments, the system may be configured to divide the data into a training data set, a testing data set, and a validation set. The system may be configured to use the training data set to perform training, and then use the testing and validation data sets to determine performance of the machine learning model.

In some embodiments, the system may be configured to determine labels for sets of input features. The system may be configured to use the determined labels to perfom a supervised learning technique. In some embodiments, the system may be configured to determine labels for sets of input features using diagnosis data (e.g., diagnosis data 144). For example, the diagnosis data may include diagnosis codes for the subjects indicating diagnoses by care providers (e.g., physicians). As an illustrative example, the system may identify diagnosis codes associated with a care provider's diagnosis of OUD and/or opioid overdose. For subjects that the diagnosis data includes diagnosis codes associated with diagnosis of OUD and/or opioid overdose (e.g., as shown in Tables 3 and 4), the system may label the corresponding input features with a classification of having a risk of OUD and/or an opioid overdose episode. The labels may represent target outputs based on which the machine learning model may be trained (e.g., by performing a supervised learning technique).

Next, process 400 proceeds to block 406, where the system obtains a machine learning model. In some embodiments, the system may be configured to obtain a machine learning model by randomly initializing parameters of the model (e.g., parameters 142 of machine learning model 140B). For example, the system may randomly initialize weights of a neural network. In another example, the system may initialize one or more coefficients of a logistic regression model. In yet another example, the system may initialize a function of the machine learning model. In some embodiments, the system may be configured to obtain a machine learning model by obtaining a previously trained machine learning model. For example, the system may obtain a machine learning model that was trained by performing process 400 using a different set of training data. The system may retrain the machine learning model using new training data instead of or in addition to the previously used training data.

After obtaining a machine learning model at block 406, process 400 proceeds to perform an iterative training procedure 407. The system may be configured to perform iterative training steps at blocks 408 to 414 to obtain a machine learning model with learned parameters (e.g., machine learning model 102B).

The iterative training procedure 407 begins at block 408, where the system determines a prediction for subjects using the machine learning model obtained at block 406. The system may be configured to determine the prediction by providing input features generated for each subject as input to the machine learning model to obtain a corresponding output. For example, the system may obtain a classification for each of the subjects indicating a prediction of risk of OUD and/or of an opioid overdose episode. In another example, the system may obtain, for each subject, a predicted likelihood (e.g., probability) that the subject will develop OUD and/or have an opioid overdose episode. The system may be configured to provide input features for a subject as input to the machine learning model to obtain an output as described at block 206 of process 200 described herein with reference to FIG. 2.

Next, process 400 proceeds to block 410, where the system determines a difference between target labels and the prediction determined at block 408. The system may be configured to determine, for each subject, a difference between a classification predicted by the machine learning model and one indicated by a target label. In some embodiments, the system may be configured to use the difference between the target labels and the prediction to determine a cost or loss function. The system may be configured to use the cost or loss function in iterative training procedure 407 to adjust parameters of the machine learning model. For example, the system may determine a mean squared error (MSE), mean absolute error (MAE), cross-entropy loss function, elastic net (EN) regularization cost function, L1 loss function, or L2 loss function using the difference between the target labels and the prediction.

Next, process 400 proceeds to block 412, where the system updates the machine learning model based on the difference between the target labels and the prediction. In some embodiments, the system may be configured to update the machine learning model by updating parameters of the machine learning model. For example, the system may update weights of a neural network. In another example, the system may update coefficients of a logistic regression model. In some embodiments, the system may be configured to use stochastic gradient descent to update the parameters of the machine learning model. In this example, the system may determine a partial derivative of a cost or loss function (e.g., cross-entropy loss function or EN regularization cost function) with respect to each parameter, and then update each parameter based on its partial derivative. The system may update each parameter by subtracting a proportion of the partial derivative from the parameter. The proportion may be configurable to adjust a learning rate of the training.

In some embodiments, the system may be configured to perform gradient boosting. The system may be configured to update the machine learning model by adding a new model to the machine learning model. The system may be configured to obtain the new model by training the new model on the difference between the target labels and the prediction. As an illustrative example, the machine learning model may be a first decision tree fit to the training data. The new model may be a second decision tree trained on the difference between the target labels and the prediction. The system may sum the first decision tree with the second decision tree to obtain an updated decision tree.

Next, process 400 proceeds to block 414, where the system determines whether the training procedure 407 has converged. In some embodiments, the system may be configured to determine whether the training procedure 407 has converged by determining whether a threshold number of iterations have been performed. For example, the system may determine that the training procedure 407 has converged when 10, 50, 100, 200, 400, 400, 500, or 1,000 iterations have been performed. In some embodiments, the system may be configured to detennine whether the training procedure 407 has converged using the difference between the target labels and the prediction. For example, the system may determine whether the training procedure 407 has converged based on whether a cost or loss function is less than a threshold value. If, at block 414, the system determines that the training procedure 407 has not converged, then process 400 proceeds to block 408, where the system uses the updated machine learning model to determine a prediction. If, at block 414, the system determines that the system has converged, then process 400 proceeds to block 416, where the system determines a decision threshold and/or a stratification.

In some embodiments, the decision threshold may be used to determine a prediction for a subject based on the output of the trained machine learning model. For example, if an output of the trained machine learning model is greater than the decision threshold, then the system may be predicted to be at risk for OUD and/or an opioid overdose episode in a time period (e.g., in 3 months, 6 months, or 9 months). In some embodiments, the output of the machine learning model may be a likelihood (e.g., a probability) that a subject will develop OUD and/or have an opioid overdose episode in a time period.

In some embodiments, the system may be configured to determine a decision threshold that optimizes a measure of performance of the machine learning model. For example, the system may select the decision threshold that optimizes the Youden index on a set of test data. In another example, the system may select the decision threshold to achieve a level of sensitivity, specificity, positive predictive value (PPV), negative predictive value (NPV), positive likelihood ratio (PLR), negative likelihood ratio (NLR), number needed to evaluate (NNE) to identify OUD and/or opioid overdose, overall misclassification rate, F1 score, C-statistic, precision-recall curve, and/or the estimated rate of generated alerts of the machine learning model.

In some embodiments, a stratification may be used to categorize a subject into one of different levels of risk. An output of the machine learning model may be used to categorize the subject into one of a number of risk levels. The system may be configured to determine the stratification by determining multiple boundary values of the machine learning model defining different risk levels. For example, the system may determine the different risk levels to be deciles of a likelihood (e.g., probability) output by the machine learning model. In another example, the system may determine the different risk levels to be quartiles of a likelihood output by the machine learning model.

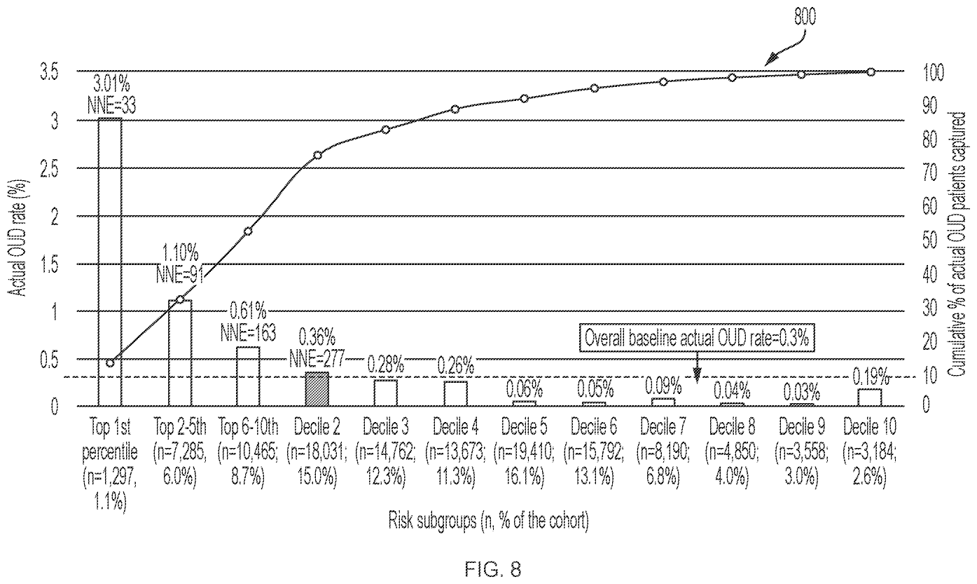

FIG. 8 is a plot 800 of a distribution of subjects based on predicted risk of OUD by a machine learning model, according to some embodiments of the technology described herein. The plot 800 displays the actual OUD rate for subjects in each decile of a predicted likelihood (e.g., probability) of the machine learning model. For the top decile, the plot 800 displays the actual OUD rate for subjects in the top 15^{th} percentile, the top 2-5^{th} percentile, and the top 6^{th}-10^{th} percentile of the top decile. As shown in FIG. 8, subjects with a predicted likelihood in the top 1′ percentile have an actual OUD rate of 3.01%, while subjects with a predicted likelihood in the 3^{rd}-10^{th} deciles have an actual OUD rate of less than 0.3%, which is the baseline OUD rate among all the subjects. In some embodiments, the system may be configured to select a decision threshold at block 418 based on resources. For example, if a medical facility has fewer resources to treat subjects predicted OUD and/or opioid overdose, the medical facility may choose to select a higher decision threshold (e.g., requiring the likelihood to be in the 15^{th} percentile) as the machine learning model is less likely to predict that a subject is at risk. Whereas, if a medical facility has greater resources to treat subjects with predicted OUD and/or opioid overdose, the medical facility may choose a lower decision threshold (e.g., a likelihood in the 2′ decile) as the machine learning model is more likely to predict that a subject is at risk. In some embodiments, the likelihood outputted by the machine learning model may be used to stratify a subject into one of multiple risk groups (e.g., low, medium, or high). For example, a subject in the top 1^{st} percentile may be deemed a high risk and thus be provided with more significant interventions than a subject in the 6^{th} to 10^{th} deciles who may be deemed a low risk. A threshold for each risk group may be configurable by a user of the machine learning model (e.g., a medical facility). As an illustrative example, for an EN regularized logistic regression model, a threshold of 0.42 may be used to optimize for the Youden index. In some embodiments, a threshold may be selected based on a percentile of predicted likelihoods. For example, a threshold of 0.95 may represent the top 1 percent of predicted likelihoods, a threshold of 0.77 may represent the top 5 percent of predicted likelihoods, and 0.61 may represent the top 10 percent of predicted likelihoods.

Although the example of FIG. 8 shows a plot 800 of an example distribution of subjects based on predicted risk of OUD by a machine learning model, in some embodiments, the plot 800 may be a distribution of a predicted risk of an opioid overdose episode. The plot 800 would be actual rate of an opioid overdose episode for subjects in each decile of a predicted likelihood (e.g., probability) of the machine learning model.

After determining a decision threshold at block 416, process 400 proceeds to block 418, where the system evaluates performance of the machine learning model. The system may be configured to evaluate performance of the machine learning model using one or more measures of performance. For example, the system may evaluate performance of the machine learning model by determining one or more of sensitivity, specificity, positive predictive value (PPV), negative predictive value (NPV), positive likelihood ratio (PLR), negative likelihood ratio (NLR), number needed to evaluate (NNE), overall misclassification rate, F1 score, C-statistic, precision-recall curve, and/or the estimated rate of generated alerts of the machine learning model. Table 5 below shows performance results in predicting OUD for various different machine learning models trained using techniques of some embodiments with decision thresholds optimized based on the Youden index and maximizing PPV.

TABLE 5
Performance Results for Example Machine Learning Models

| | Score | | | | | | | | |
|---|---|---|---|---|---|---|---|---|---|
| | threshold | | | | | | F1 | | |
| | (range | Predicted | Sensitivity | Specificity | PPV | NPV | score | | |
| Methods | 0-100) ^{a} | OUD (%) | (%) | (%) | (%) | (%) | (%) | PLR | NNE |
| Elastic Net | | | | | | | | | |
| Optimized | 42.25 | 21.57 | 81.49 | 78.51 | 0.54 | 99.97 | 0.0108 | 3.79 | 184 |
| Youden Index | | | | | | | | | |
| Threshold | | | | | | | | | |
| Maximized | 99.82 | 0.00 | 0.05 | 100.00 | 33.33 | 99.86 | 0.0011 | 347.30 | 3 |
| PPV | | | | | | | | | |
| GBM | | | | | | | | | |
| Optimized | 46.01 | 19.67 | 80.42 | 80.42 | 0.59 | 99.96 | 0.0117 | 4.11 | 170 |
| Youden Index | | | | | | | | | |
| Threshold | | | | | | | | | |
| Maximized | 95.48 | 0.00 | 0.11 | 100.00 | 16.67 | 99.86 | nan | 0.00 | Inf |
| PPV | | | | | | | | | |
| RF | | | | | | | | | |
| Optimized | 46.16 | 21.28 | 80.26 | 78.81 | 0.54 | 99.96 | 0.0108 | 3.79 | 184 |
| Youden Index | | | | | | | | | |
| Threshold | | | | | | | | | |
| Maximized | 94.23 | 0.01 | 0.37 | 99.99 | 6.73 | 99.86 | 0.0071 | 50.13 | 15 |
| PPV | | | | | | | | | |
| DNN | | | | | | | | | |
| Optimized | 40.10 | 19.22 | 79.13 | 80.87 | 0.59 | 99.96 | 0.0118 | 4.14 | 169 |
| Youden Index | | | | | | | | | |
| Threshold | | | | | | | | | |
| Maximized | 99.26 | 0.00 | 0.05 | 100.00 | 50.00 | 99.86 | 0.0011 | 694.59 | 2 |
| PPV | | | | | | | | | |

Table 6 below shows a comparison of prediction performance of machine learning techniques of some embodiments to Centers for Medicaid and Medicaid Services (CMS) high-risk opioid use measures. In particular, performance of deep neural network (DNN), and GBM models is compared to the CMS measures in a sample of subjects over a 12-month period. The results in Table 6 show that the DNN and GBM models outperform CMS measures. The CMS measures were based on a 12-month period rather than 3 months. If classifying beneficiaries with any of CMS high-risk opioid use measures as OUD, the remaining may be considered as non-OUD. CMS Opioid safety measures, which are meant to identify high-risk individuals or utilization behavior, may include any of the following 3 metrics: (1) high-dose use, defined as >120 MME for >90 continuous days, (2)>4 opioid prescribers and >4 pharmacies, (3) concurrent opioid and benzodiazepine use >30 days. In the example of Table 6, the DNN and GBM models have different prediction probability distributions: individuals with (1) predicted probability in the top 1 percentile (DNN=0.93, and GBM=0.90); (2) predicted probability in the top 2nd to 5th percentile (DNN=0.76, and GBM=0.72); and (3) predicted probability in the top 6th to 10th percentile (DNN=0.6, and GBM=0.59). For each model, Table 6 shows the performance results when the threshold probability value to classify a subject as being athigh risk of OUD is set to the top 1 percentile threshold, the top 5th percentile threshold, and the top 10th percentile threshold.

TABLE 6
Performance Results Relative to CMS Measures

| | Any CMS | | High risk defined as using different | | | High risk defined as using | | |
|---|---|---|---|---|---|---|---|---|
| | measures | | DNN's thresholds | | | different GBM's thresholds | | |
| | Low | High | Top 1 | Top 5^{th} | Top 10^{th} | Top 1 | Top 5^{th} | Top 10^{th} |
| Risk | risk | Risk | percentile | percentile | percentile | percentile | percentile | percentile |
| subgroups | (n = | (n = | (n = | (n = | (n = | (n = | (n = | (n = |
| (n, % of the | 110,171, | 4,082, | 2,213, | 11,093, | 20,654, | 2,115, | 11,211, | 22,232, |
| cohort | 96.4%) | 3.6% | 1.9%) | 9.7%) | 18.1%) | 1.9%) | 9.8% | 19.5%) |
| Number of | 412 | 115 | 187 | 378 | 452 | 173 | 373 | 468 |
| actual OUD | (0.37) | (3.80 | (8.5) | (3.41) | (2.19) | (8.18) | (3.33) | (2.11) |
| (% of each | | | | | | | | |
| subgroup | | | | | | | | |
| Number of | 109,759 | 3,927 | 2,026 | 10,715 | 20,202 | 1,942 | 10,838 | 21,764 |
| actual non- | (99.6) | (96.2) | (91.55) | (96.59) | (97.81) | (91.82) | (96.67) | (97.89) |
| OUD (% of | | | | | | | | |
| each | | | | | | | | |
| subgroup) | | | | | | | | |
| Number | 270 | 26 | 11 | 29 | 45 | 12 | 30 | 47 |
| Needed to | | | | | | | | |
| Evaluate | | | | | | | | |
| (NNE) | | | | | | | | |
| Overall | 412 | 3,927 | 2,026 | 10,715 | 20,202 | 1.942 | 10,838 | 21,764 |
| number | (0.36) | (3.4) | (1.8) | (9.4) | (17.7) | (1.7) | (9.5) | (19.1) |
| misclassified | | | | | | | | |
| (% of overall | | | | | | | | |
| cohort) | | | | | | | | |
| % of all | 72.7 | 27.3 | 33.0 | 66.7 | 79.7 | 20.5 | 65.8 | 82.5 |
| OUD over | | | | | | | | |
| 12 months | | | | | | | | |
| (n = 567) | | | | | | | | |
| captured | | | | | | | | |

In some embodiments, the system may be configured to generate a report of performance that complies with the Standards for Reporting of Diagnostic Accuracy (STARD). In some embodiments, the system may be configured to generate a report of performance that complies with the Transparent Reporting of Multivariable Prediction Model for Individual Prognosis or Diagnosis (TRIPOD) reporting guidelines. As an illustrative example, the system may determine a C-statistic using a validation sample data set to assess discrimination. The discrimination may be the extent to which patients predicted with higher likelihoods exhibithigher OUD and/or overdose rates compared to those predicted as low risk.

In some embodiments, the system may be configured to determine predictors to use for the machine learning model from a set of candidate predictors (e.g., shown in Table 1 and Table 2). The system may be configured to select the predictors from the set of candidate predictors based on a measure of influence of each predictor on a prediction. For example, the system may determine the predictors based on an absolute value of coefficients for respective predictors in a logistic regression model. The system may be configured to adjust the machine learning model to use the selected set of predictors. For example, for a logistic regression model, the system may reduce coefficients associated with predictors thathave not been selected to 0 to obtain a logistic regression model that uses the selected predictors. Table 7 below shows an example list of 25 EN Regularization Model predictors, 25 GBM model OUD risk predictors, and 25 GBM model opioid overdose risk predictors.

TABLE 7
Example Sets of Predictors

| 1 | 2 | 3 |
|---|---|---|
| EN Regularization | GBM Model OUD Risk | GBM Model Opioid Overdose |
| Model Predictors | Predictors | Episode Predictors |
| State | Age | Age |
| Schedule IV Short-Acting Opioids | Total MME | Total MME |
| Elixhauser Drug Abuse (excluding OUD) | Lower Back Pain | Lower Back Pain |
| Lower Back Pain | Average MME Prescribed by | Average Number of Monthly |
| | Provider Per Patient | Non-Opioid Prescription |
| Race | Average Number of Monthly Non- | Number of Opioid Prescribers |
| | Opioid Prescription | |
| Non-opioid Drug Use Disorders | Number of Opioid Prescribers | Average MME Prescribed by |
| | | Provider Per Patient |
| Alcohol Use Disorder | Regional-Level: Percent | Without Disabled Eligibility |
| | Unemployment | |
| Disabled Eligibility Category | Without Disabled Eligibility | Regional-Level: Preventable |
| | | Hospitalization Rate |
| Fibromyalgia | Regional-Level: Number of | Had Urine Drug Test |
| | Medical Prescription Drug Plan | |
| | Enrollees | |
| Elixhauser Alcohol Abuse Indicator | Had Urine Drug Test | Number of ED Visits |
| Had a Urine Drug Test | Area Deprivation Index | Regional-Level: Percent |
| | | Uninsured Adults |
| Rheumatoid Arthritis | Regional-Level: Sexual | Regional-Level: Sexual |
| | Transmission Infection Rate | Transmission Infection Rate |
| Liver Disease | Regional-Level: Percent Uninsured | Regional-Level: Number of |
| | Adults | Medical Prescription Drug Plan |
| | | Enrollees |
| Resided in Metropolitan County | Regional-Level: Preventable | Regional Level: Percent |
| | Hospitalization Rate | Unemployment |
| Receiving Medicare Part D Low Income | Regional-Level: Percent People | Area Deprivation Index |
| Subsidy | Aged 18-64 without Health | |
| | Insurance | |
| Neuropathies | Regional-Level: Percent | Regional-Level: Emergency |
| | Mammography Screening | Department (ED) visits per |
| | | 1000 FFS Medicare |
| | | Beneficiaries |
| Anxiety Disorder | Regional-Level: Percent Dual | Average Daily MME |
| | Eligible Beneficiaries | |
| Male | Average Daily MME | Regional-Level: Percent People |
| | | Aged 18-64 without Health |
| | | Insurance |
| Diabetes with Complications | Regional-Level: Emergency | Schedule IV Short-Acting |
| | Department (ED) visits per 1000 | Opioids |
| | Fee-For-Service (FFS) Medicare | |
| | Beneficiaries | |
| Seen by Prescriber with 2 or more | Number of ED Visits | Regional-Level: Percent People |
| Specialties | | Not Proficient in English |
| Congestive Heart Failure | Regional-Level: Percent People | Regional-Level: Percent |
| | Not Proficient in English | Mammography Screening |
| Mood Disorders | Regional-Level: Percent Poor to | Regional-Level: Percent Poor to |
| | Fair Health | Fair Health |
| Resided in a Country Under a Persistent | Cumulative 30-Day Duration of | Elixhauser Index |
| Poverty Level | Opioid Use | |
| Musculoskeletal/Connective Tissue Disease | Schedule IV Short-Acting Opioids | Regional-Level: Percent Low |
| | | Birth Weight |
| Headache and Migraine Diagnosis | Percent Beneficiaries Enrolled in | Regional-Level: Vehicle/Crash |
| | Medicare Advantage Plans | Related Death Rate |

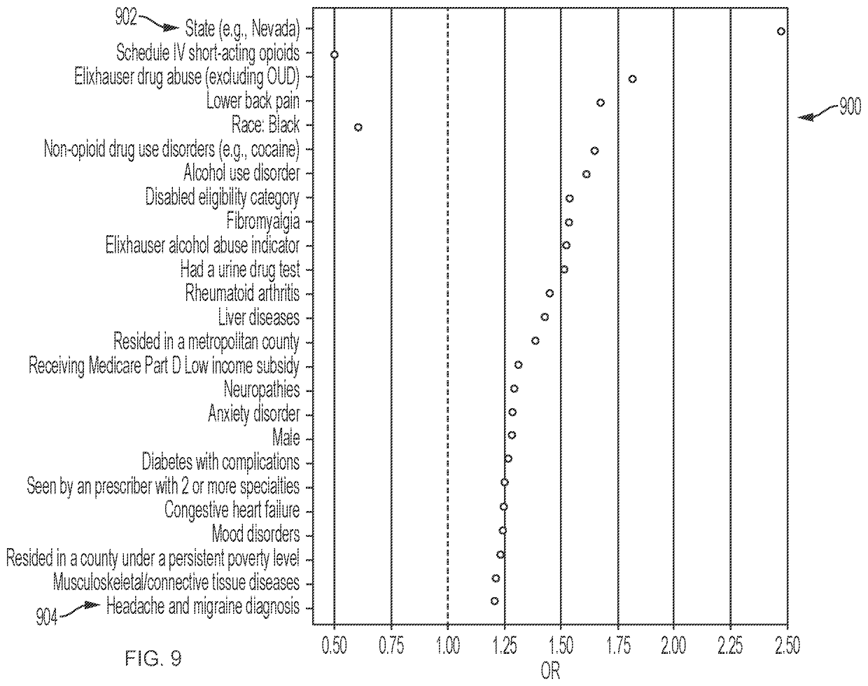

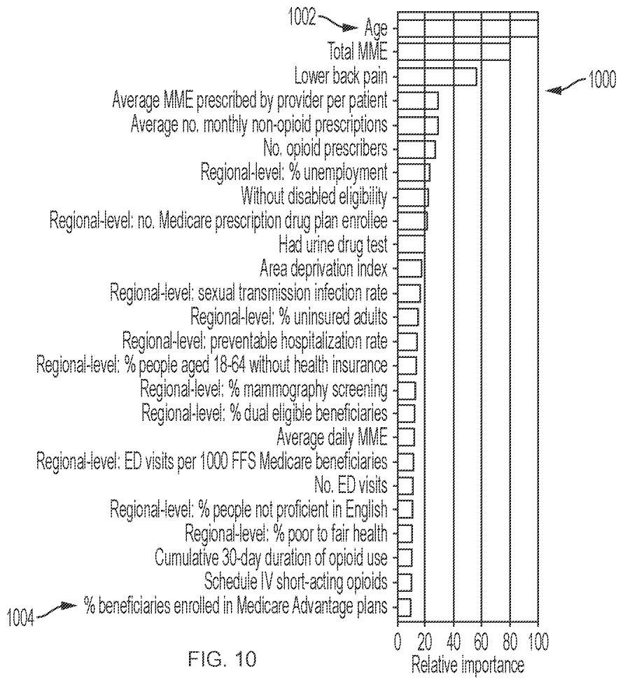

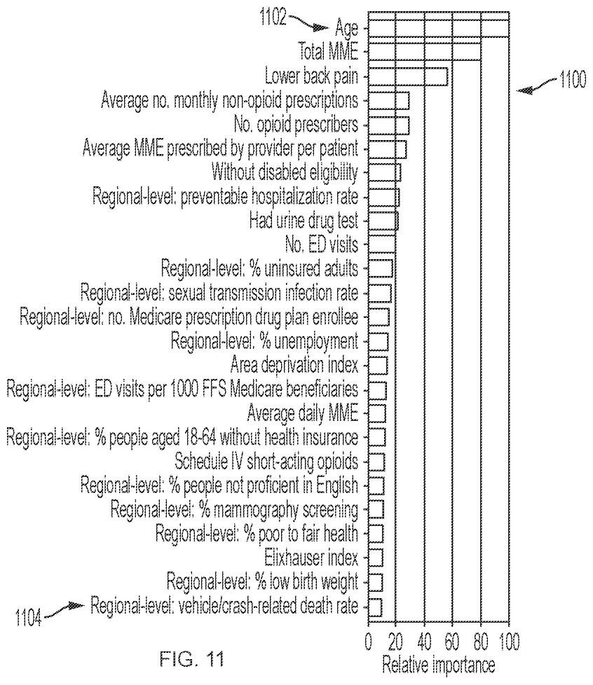

FIG. 9 is a plot 900 of an example set of top 25 predictors identified for prediction of risk of OUD in a machine learning model trained using EN regularization listed in column 1 of Table 7, according to some embodiments of the technology described herein. The plot shows an odds ratio for each of the top 25 predictors. Of the top 25 predictors shown in plot 900, the subject's state has the greatest odd ratio of approximately 2.5 while headache and migraine diagnosis have the lowest odds ratio of approximately 1.2. FIG. 10 is a plot 1000 of an example set of top 25 predictors for predicting risk of developing OUD in a subject determined by a gradient boosting machine (GBM) listed in column 2 of Table 7, according to some embodiments of the technology described herein. The plot 1000 shows a relative variable importance for each of the top 25 predictors. For example, the age predictor 1002 has the greatest variable importance of approximately 100, while the percentage of beneficiaries enrolled in Medicare Advantage plans predictor 1004 has the lowest variable importance of approximately 15. FIG. 11 is a plot 1000 of a set of an example set of top 25 predictors for predicting risk of OUD or an opioid overdose episode as determined by a gradient boosting machine (GBM) listed in column 3 of Table 7, according to some embodiments of the technology described herein. The plot 1100 shows a variable importance of each of the 25 predictors. The age predictor 1102 has the greatest variable importance of approximately 100, while the regional level of vehicle//crash-related death rate predictor 1104 has the lowest variable importance of approximately 17.

### Example Machine Learning Model Training Implementations

In some embodiments, a machine learning model for predicting risk of OUD and/or opioid overdose may be an RF model. For example, the “Random Forests Tree Ensembles” in the software package SALFORD PREDICTIVE MODELER (SPM) may be used to train the RF model. In another example, a PYTHON random forests library may be used to train the RF model. The number of trees to be included in the RF model may be 25, 50, 75. 100, 125, 150, 175, 200, 225, 250, 275, or 300 trees. The number of predictor candidates for each node is determined as the square root of the number of total predictors. For example, the number of predictor candidates at each node may be a square root of 269, which is the number of candidate predictors in Table 1. A balanced class weight function and an out of bag (OOB) function may be used during training. An example decision threshold for an RF model is 0.62, which may be identified using the Youden index.

In some embodiments, the machine learning model may be a GBM model. A software package may be used to train the GBM model. For example, a TreeNet function from SPM may be used to supply an initial value to a chosen loss function for each training sample. TreeNet may be used to handle missing values. As another example, the PYTHON XGBoost package may be used supply an initial value to a chosen loss function for each training sample. A cross entropy (e.g., negative average log likelihood) may be used as a tuning criterion to determine a number of trees for the models. The training system samples a portion (e.g., 25%) of the training data randomly and computes a generalized residual model for the records in the portion of the training data. The training system may sample training data points to fit a classification tree with a maximum 8 terminal nodes to the generalized residuals. The training system may update a tree based on a loss function and shrink the updated tree by the learning rate (e.g., 0.1) for overfitting protection. The steps may be repeated a number of times (e.g., 50, 100, 150 200, 250, or 300 times) to obtain a number of trees. The model may then be tested and validated using a testing and validation data set. An example decision threshold for an RF model is 0.49, which may be identified using the Youden index.

In some embodiments, the machine learning model may be a DNN. The DNN may have multiple hidden layers. For example, the DNN may have 2, 3, 4, 5, 6, 7, 8, 9, or 10 hidden layers, and 20, 30, 40, 60, 80, 100, 120, 140, 160, 180, or 200 nodes. The PYTHON 3.6 KERAS package may be used to train the DNN. Various numbers of hidden layers and nodes may be analyzed. In one example, a DNN with 2 hidden layers and 120 nodes was used. In each hidden layer an activation function (e.g., ReLU) may be used (e.g., to yield faster convergence). A sigmoid function may be applied to the output layer to generate a likelihood (e.g., probability) output of the machine learning model. A binary cross-entropy loss function with balanced class weights in the training data may be used to train the DNN (e.g., using stochastic gradient descent). A hyperparameter search may also be performed to optimize the DNN. For example, a grid search may be performed to identify L1 and L2 regularization weights. An example decision threshold for a DNN model is 0.40, which may be identified using the Youden index.

### Trajectories of Concurrent Opioid and Benzodiazepine (BZD) Use Based OUD and Opioid Overdose Prediction Techniques

One-third of opioid overdose deaths involve concurrent benzodiazepine (BZD) use. Concurrent opioid and BZD use can cause synergistic respiratory depression and can substantially increase the risk of overdose. Further, compared with opioid use alone, concurrent opioid and BZD users show a 2 to 6 fold increase opioid overdose risk. Accordingly, the inventors have developed techniques that use information about opioid and BZD dosage combination use patterns over time in a subject to predict a risk of OUD and/or an opioid overdose episode. The techniques use a statistical model to determine a longitudinal opioid-BZD dosage pattern of a subject, and determine a risk of OUD and/or an opioid overdose episode within a period of time based on the identified patterns. A longitudinal dosage pattern over time may also be referred to herein as a “pattern” or a “trajectory”. In some embodiments, the techniques estimate a time to first opioid overdose within a time period using inverse probability of treatment weighted Cox proportional hazard models.

The inventors have identified longitudinal concurrent opioid and BZD use patterns over time that may be used to predict the risk of OUD and/or an opioid overdose episode. A longitudinal concurrent opioid and BZD use pattern over time may be referred to herein as “opioid-BZD trajectory”. In some embodiments, the techniques classify a subject into one of 9 different opioid-BZD trajectories. The opioid-BZD trajectories are: (1) very low dose opioid and slowly decreasing BZD dose; (2) very low dose opioid and consistent BZD dose; (3) very low dose opioid with medium dose BZD; (4) low does opioid and BZD; (5) low does opioid with high dose BZD; (6) medium dose opioid with low dose BZD; (7) very high dose opioid with high dose BZD; (8) very high dose opioid with very high dose BZD; and (9) very high dose opioid with low dose BZD. Each category is characterized by a respective trajectory.

Some embodiments use group-based multi-trajectory modeling to identify distinct longitudinal concurrent opioid and BZD dosage patterns over time (i.e., opioid-BZD trajectories). Some embodiments use data about Medicare beneficiaries to identify the opioid-BZD trajectories. For example, the techniques use data from Medicare master beneficiary summary files, Part D drug event files, and medical claims to identify the patterns. The techniques identify the patterns by: (1) constructing daily measures of averaue standardized daily dose (SDD) separately foe opioids and BZD during a period of time after initiation of opioids; and (2) applying group-based multi-trajectory models with SDD as the model's outcome to identify distinct dose and duration patterns. In some embodiments the period of time may be 1 month, 2 months, 3 months, 4 months, 5 months, 6 months, 7 months, 8 months, 9 months, 10 months, 11 months, or 1 year.

Some embodiments calculate SDD for opioids by determining daily morphine milligram equivalents (MME) using dispensing dose, date, and days' supply. For BZD, the techniques determine diazepam milligram equivalents (DME). The opioid use is cateuorized as very low (SDD <25 MME), low (25-50 MME), moderate (51-90 MME), high (91-150 MME), and very high (>150 MME). The BZD is categorized as very low (<10 DME), low (10-20 DME), moderate (21-40 DME), high (41-60 DME), and very high (>60 DME). The techniques identify longitudinal concurrent opioid BZD use patterns based on doses used over time using group-based multi-trajectory models.

In some embodiments, the outcome used to generate the model is the amount of time to the development of OUD and/or an opioid overdose episode in a time period after data collection. For example, the outcome may be an opioid overdose episode in 1 month, 2 months, 3 months, 4 months, 5 months, 6 months, 7 months, 8 months, 9 months, 10 months, 11 months, or 1 year after a data collection period. Some embodiments identify overdose events using International Classification of Diseases codes (e.g., ICD-9/ICD-10). In some embodiments, the outcome may be time to diagnosis of BZD overdose, or a time to diagnosis of either opioid overdose or BZD overdose.

Some embodiments measure predictors during a period of time prior to opioid initiation. For example, at least some of the predictors of Table 1 and/or Table 2 may be measured for 1 month, 2 months, 3 months, 4 months, 5 months, 6 months, 7 months, 8 months, 9 months, 10 months, 11 months, or 1 year prior to opioid initiation.

Some embodiments use machine learning techniques to determine inverse probability of treatment weights (IPTW) for a subject using gradient boosting machine. IPTW is the inverse probability of a subject's likelihood to be placed in a specific opioid-BZD trajectory group. The IPTW is used as a weight to generate a sample in which assignment to a pattern is independent of measured covariates. In some embodiments, machine learning model 102B described herein with reference to FIGS. 1A-1B may be used to determine the probability. The IPTW may be determined as an inverse of the probability output by the machine learning model 102B. In some embodiments, subjects with IPTWs greater than a threshold value (e.g., 5, 10, 15, 20, 25, or 30) are excluded. Some embodiments use IPTW-weighted multivariable Cox proportional hazard models to compare time to OUD diagnosis and/or opioid overdose events.

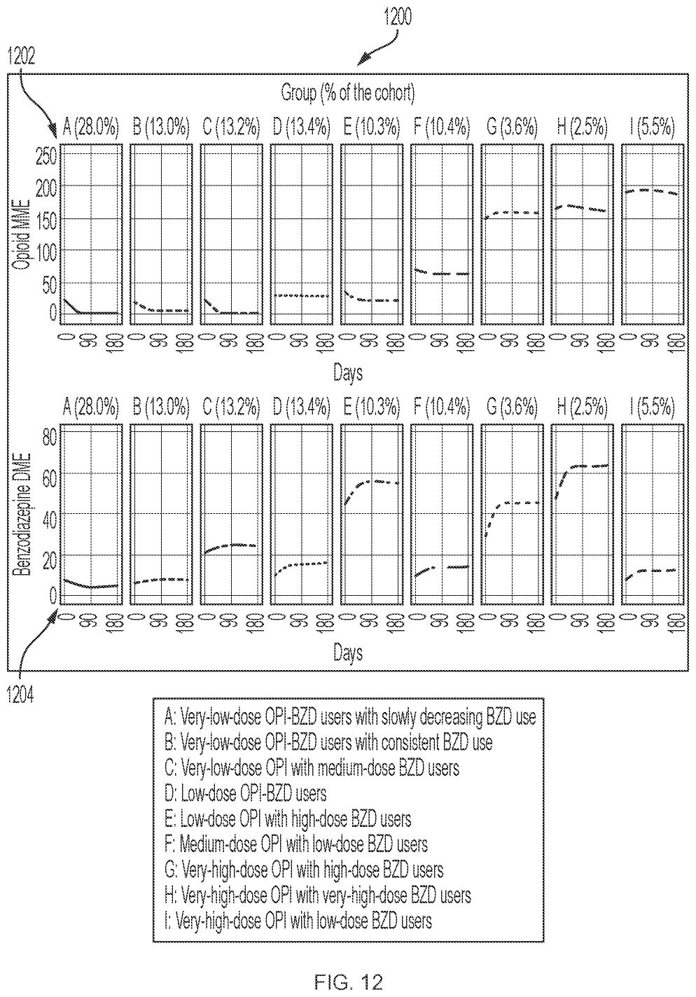

FIG. 12 is a plots 1200 of concurrent opioid and BZD use trajectories, according to some embodiments of the technology described herein. The plots 1200 includes a first set 1202 of plots showing opioid dosage over a period of 180 days for 9 patterns. The plots 1200 include a second set 1204 of plots showing BZD dosage over a period of 180 days for the 9 patterns. The opioid-BZD trajectories are described as follows: “A” very low opioid dose with a slowly decreasing BZD dose; “B” very low opioid dose with a consistent BZD dose; “C” very low opioid dose with medium BZD dose; “D” low opioid dose with low BZD dose; “E” low opioid dose with high BZD dose; “F” medium opioid dose with low BZD dose; “G” very high opioid dose with high BZD dose; “H” very high opioid dose with very high BZD dose; and “I” very high opioid dose with low BZD dose.

In some embodiments, the opioid use management system 100 described herein with reference to FIGS. 1A-1B may be configured to obtain measurements of opioid and BZD dosage of a subject over a period of time (e.g., 1 month, 2 months, 3 months, 4 months, 5 months, 6 months, 7 months, 8 months, 9 months, 10 months, 11 months, or 12 months). The system 100 may be configured to categorize the subject into one of the trajectories (e.g., illustrated in FIG. 12). The system 100 may be configured to determine a risk (e.g., low, medium, or high) of opioid overdose for the subject in a period of time according to the trajectory the subject is categorized in. For example, the system 100 may determine high risk of OUD and/or opioid overdose episode for the subject if the subject is categorized into one of opioid-BZD trajectories “E”, “F”, “G”, “H”, or “I” shown in FIG. 12. The system 100 may determine low risk of opioid overdose for the subject if the subject is categorized into one of opioid-BZD trajectories “A”, “B”, or “C” shown in FIG. 12. The system may determine a medium risk of opioid overdose for the subject if the subject is categorized into pattern “E”.

In some embodiments, the user interface module 108 of the system 100 may be configured to generate a GUI indicating a risk level of OUD/and/or an opioid overdose episode based on the determined risk. As shown in GUI 500 of FIG. 5, the GUI 500 indicates a BZD medication 510A for patient “Doe, Jone” 502A and BZD medication 510B for patient “Smith, Mary” 502B. The GUI 500 further indicates dosages 512A, 512B for each of the subjects 502A, 502B. In some embodiments, the overdose risks 504A, 504B for each of the subjects 502A, 502B may be determined by the system 100 based on the pattern that the subject is categorized into (e.g., based on collected dosage measurement data).

### Example Computer System

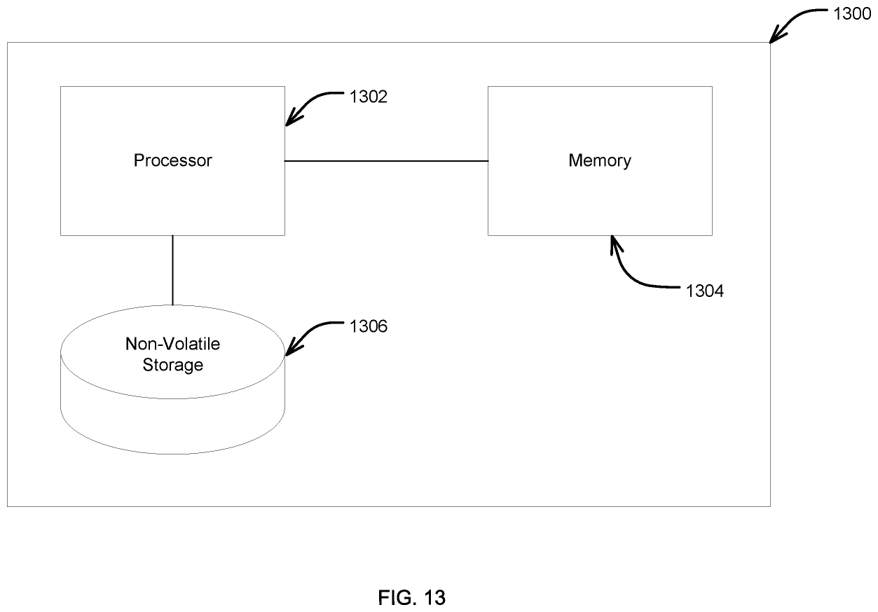

FIG. 13 is a diagram of an example computer system 1300, according to some embodiments of the technology described herein. Computer system 1300 may be used to implement some embodiments of the technology described herein. The computing system 1300 may include one or more computer hardware processors 1302 and non-transitory computer-readable storage media (e.g., memory 1304 and one or more non-volatile storage devices 1306). The processor(s) 1302 may control writing data to and reading data from (1) the memory 1304; and (2) the non-volatile storage device(s) 1306. To perform any of the functionality described herein, the processor(s) 1302 may execute one or more processor-executable instructions stored in one or more non-transitory computer-readable storage media (e.g., the memory 1304), which may serve as non-transitory computer-readable storage media storing processor-executable instructions for execution by the processor(s) 1302.

It should be appreciated that the techniques introduced above and described in greater detail below may be implemented in any of numerous ways, as the techniques are not limited to any particular manner of implementation. Examples of details of implementation are provided herein solely for illustrative purposes. Furthermore, the techniques disclosed herein may be used individually or in any suitable combination, as aspects of the present disclosure are not limited to the use of any particular technique or combination of techniques.

Having thus described several aspects of at least one embodiment, it is to be appreciated that various alterations, modifications, and improvements will readily occur to those skilled in the art. Such alterations, modifications, and improvements are intended to be within the spirit and scope of the present disclosure. Accordingly, the foregoing description and drawings are by way of example only.

The above-described embodiments of the present disclosure can be implemented in any of numerous ways. For example, the embodiments may be implemented using hardware, software, or a combination thereof When implemented in software, the software code can be executed on any suitable processor or collection of processors, whether provided in a single computer or distributed among multiple computers. Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure described above. The computer-readable medium or media can be transportable, such that the program or programs stored thereon can be loaded onto one or more different computers or other processors to implement various aspects of the present disclosure as described above.

The terms “program” or “software” are used herein to refer to any type of computer code or set of computer-executable instructions that can be employed to program a computer or other processor to implement various aspects of the present disclosure as described above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Various features and aspects of the present disclosure may be used alone, in any combination of two or more, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim elementhaving a certain name from another elementhaving a same name (but for use of the ordinal term) to distinguish the claim elements.

The indefinite articles “a” and “an,” as used herein in the specification and in the claims, uncles clearly indicated to the contrary, should be understood to mean “at least one.”

The phrase “and/or” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Thus, as an example, a reference to “A and/or B” can refer to: (1) one embodiment including A without B; (2) another embodiment including B without A; and (3) another embodiment including both A and B.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

1. A method for using a trained machine learning model to predict risk of incident opioid use disorder (OUD) and/or of an opioid overdose episode for a subject, the method comprising:
using at least one computer hardware processor to perform:
accessing data associated with the subject, wherein the data comprises values for at least 10 predictors from among predictors shown in Table 1 and/or Table 2;
generating input features for the trained machine learning model from the data; and
providing the input features as input to the trained machine learning model to obtain an output indicative of the risk of OUD and/or the opioid overdose episode for the subject,
wherein the trained machine learning model comprises a first plurality of values for a respective first plurality of parameters, the first plurality of values used by the at least one computer hardware processor to obtain the output from the input features.

2. The method of claim 1, wherein the trained machine learning model comprises a logistic regression model.

3. The method of claim 2, wherein the logistic regression model is trained using a regularization technique.

4. The method of claim 3, wherein the regularization technique is Elastic Net regularization.

5. The method of claim 1, further comprising training a machine learning model using training data and a supervised learning to technique to obtain the trained machine learning model, wherein the training data comprises paired data comprising input-output pairs, each input-output pair having input values for the at least 10 predictors and a corresponding output value indicative of a risk of OUD and/or the opioid overdose episode, wherein the corresponding output value indicative of the risk of OUD is set based on an indication of OUD diagnosis, and/or initiation of methadone or buprenorphine.

6. (canceled)

7. The method of claim 5, wherein a corresponding output value indicative of the risk of the opioid overdose episode is set based on an indication of an opioid overdose episode diagnosis.

8. The method of claim 1, wherein the trained machine learning model comprises a deep neural network model, a random forest model, and/or a gradient boosting machine model.

9. The method of claim 1, wherein the output from the trained machine learning model indicates the risk of OUD and/or the opioid overdose episode for the subject within 3 months of the subject receiving an opioid prescription.

10-21. (canceled)

22. The method of claim 1, wherein the output of the trained machine learning model is indicative of the risk of the opioid overdose episode for the subject, and wherein the data comprises values for a predictor indicating whether the subject has a previous history of OUD and/or an opioid overdose episode.

23. (canceled)

24. The method of claim 1, further comprising,
determining whether to intervene with the subject based on the output indicative of the risk of OUD and/or the opioid overdose episode for the subject; and
in response to determining to intervene with the subject, selecting the subject for enrollment in a lock-in program, making an outreach call to the subject, referring the subject to a use disorder specialist, prescribing an opioid antagonist therapy, administering an opioid antagonist therapy to the subject, and/or initiating an evidence-based intervention.

25. (canceled)

26. The method of claim 25, wherein
initiating the evidence-based intervention comprises initiating use of medication used to treat OUns wherein the medication comprises buprenorphine and/or naltrexone.

27. (canceled)

28. The method of claim 25, further comprising:
in response to determining to intervene with the subject, prescribing and/or administering an opioid antagonist therapy to the subject, wherein the opioid antagonist therapy comprises naloxone.

29-30. (canceled)

31. The method of claim 1, wherein the data associated with the subject comprises information about concurrent opioid and benzodiazepine (BZD) use by the subject, and the method further comprises predicting the risk of OUD and/or the opioid overdose episode for the subject using the information about the concurrent opioid and BZD use by the subject.

32. The method of claim 31, wherein predicting the risk OUD and/or the opioid overdose episode for the subject using the information about the concurrent opioid and BZD use by the subject comprises determining a longitudinal opioid-BZD dosage pattern over time of the subject.

33. The method of claim 31, wherein predicting the risk of OUD and/or the opioid overdose episode for the subject using the information about concurrent opioid and BZD use by the subject comprises generating at least one of the input features for the trained machine learning model using the information about concurrent opioid and BZD use by the subject.

34. The method of claim 33, wherein predicting the risk of OUD and/or the opioid overdose episode for the subject using the information about concurrent opioid and BZD use by the subject comprises:
determining an opioid-BZD trajectory of the subject using the information about the concurrent BZD and opioid use by the subject; and
predicting the risk of OUD and/or the opioid overdose episode based on the opioid-BZD trajectory.

35. The method of claim 34, wherein determining the opioid-BZD trajectory of the subject comprises selecting one of a plurality of predetermined opioid-BZD trajectories.

36. The method of claim 35, wherein the plurality of predetermined opioid-BZD trajectories consist of 9 trajectories, wherein the 9 trajectories are: very low opioid dose with a slow decreasing BZD dose, a very low opioid dose with a consistent BZD dose, a very low opioid dose with a medium BZD dose, a low opioid dose with a low BZD dose, a low opioid dose with a high BZD dose, a medium opioid dose with a low BZD dose, a very high opioid dose with a high BZD dose, a very high opioid dose with a very high BZD dose, and a very high opioid dose with a low BZD dose.

37-40. (canceled)

41. A system for using a trained machine learning model to predict risk of incident opioid use disorder (OUD) and/or of an opioid overdose episode for a subject, the system comprising:
at least one computer hardware processor; and
at least one non-transitory computer-readable storage medium storing instructions that, when executed by the at least one computer hardware processor, cause the at least one computer hardware processor to perform:
accessing data associated with the subject, wherein the data comprises values for at least 10 predictors from among predictors shown in Table 1 and/or Table 2;
generating input features for the trained machine learning model from the data; and
providing the input features as input to the trained machine learning model to obtain an output indicative of the risk of OUD and/or the opioid overdose episode for the subject,
wherein the trained machine learning model comprises a first plurality of values for a respective first plurality of parameters, the first plurality of values used by the at least one computer hardware processor to obtain the output from the input features.

42. At least one non-transitory computer-readable storage medium storing instructions that, when executed by at least one computer hardware processor, cause the at least one computer hardware processor to perform:
accessing data associated with a subject, wherein the data comprises values for at least 10 predictors from among predictors shown in Table 1 and/or Table 2;
generating input features for a trained machine learning model from the data; and
providing the input features as input to the trained machine learning model to obtain an output indicative of a risk of OUD and/or of an opioid overdose episode for the subject,
wherein the trained machine learning model comprises a first plurality of values for a respective first plurality of parameters, the first plurality of values used by the at least one computer hardware processor to obtain the output from the input features.

43-55. (canceled)
